(19) United States
(12) Patent Application Publication
(10) Pub. No.: US 2021/0102188 A1
(43) Pub. Date: Apr. 8, 2021

(54) Production and Therapeutic Uses of Epinul Cells and Differentiated Cells Derived Therefrom

(71) Applicant: K2 Research Holdings, LLC, West Hempstead, NY (US)

(72) Inventor: Daniel Kilbank, Bethesda, MD (US)

(21) Appl. No.: 17/125,447

(22) Filed: Dec. 17, 2020

Related U.S. Application Data
(63) Continuation of application No. 15/805,020, filed on Nov. 6, 2017, now abandoned.
(60) Provisional application No. 62/418,304, filed on Nov. 7, 2016.

(51) Int. Cl. C12N 13/00; C12N 5/074; A61K 35/545; A61K 35/28; C12N 5/0735; C12N 5/0793

(52) U.S. Cl. CPC C12N 13/00; C12N 2529/00; A61K 35/545; A61K 35/28; C12N 5/0606; C12N 5/0619; C12N 2501/727; C12N 2501/115; C12N 2500/38; C12N 2501/235; C12N 2501/105; C12N 2501/11; C12N 2501/155; C12N 2501/16; C12N 2501/165; C12N 2506/1307; C12N 2521/10; C12N 5/0696

(57) ABSTRACT

Compositions and methods are provided for the generation of highly potent conditioned stem (Epinul) cells from adult somatic cells or tissues. Such conditioned stem cells are capable of generating all the cell lineages of any tissue or organ. Uses and compositions of the conditioned stem cells are also disclosed.

### RELATED APPLICATION DATA

This application is a continuation of U.S. patent application Ser. No. 15/805,020, filed Nov. 6, 2017, which application claims the benefit of priority of U.S. Provisional Patent Application Ser. No. 62/418,304, filed Nov. 7, 2016, and titled “Production and Therapeutic Uses of Synthetically Generated Somatic Stem Cells”. Each application is incorporated by reference herein in its entirety.

### FIELD

The present disclosure relates to the fields of stem cell biology and regenerative medicine, and more particularly to compositions and methods of developing and artificially producing clinically viable stem cells for medical applications, such as diagnosis, screening, testing, therapy, and rehabilitation, as well as cells for use in commercial applications, such as screening, testing, and bioengineering.

### BACKGROUND

Stem cells provide new regenerative opportunities for patients with cancer, spinal cord injuries, stroke, degenerative diseases, and other conditions, because of their plasticity and potential use to replace diseased, injured or aged tissues and organs. It has been suggested that by using stem cell transplants instead of drugs, biologics, and other current therapies, stem cells can offer new therapies for the prevention and/or treatment of various human disorders and conditions.

Embryonic stem (ES) cells are believed to have particular promise due to their pluripotent nature, i.e. the ability to replicate indefinitely and differentiate into cells of all three germ layers (Thomson et al., Science 282:1145-1147 (1998), incorporated by reference herein in its entirety). The application of human ES cells in therapy and regenerative medicine is complicated, however, by the possibility of rejection by the recipient's immune system. Human pluripotent cells that are substantially genetically identical to a particular recipient are, thus, highly desirable. Also, genetic identity may be important for the use of ES cells in designing patient-specific treatment strategies.

Initial attempts to generate pluripotent cells from a post-natal primate individual employed somatic nuclear transfer (see, e.g., Byrne, J A et al., Nature 450:497-502 (2007)) and cell fusion (see, e.g., Yu, J et al., Stem Cells 24:168-176 (2006)). However, clinical use of somatic nuclear transfer is impractical due to its low efficiency, while cell fusion results in near tetraploid cells. In 2007, two groups of scientists reprogrammed somatic cells from a post-natal primate individual into pluripotent stem cells (Yu et al. Science 318:1917-1920 (2007) and Takahashi et al., Cell 131:861-872 (2007)), each incorporated by reference herein in its entirety. Both groups delivered into, and expressed in, human somatic cells cDNA of four transcription factors using a viral vector system for expressing potency-determining transgenes. The transcription factors of Takahashi et al. were OCT4, SOX2, c-Myc, and KLF4, while Yu et al. employed OCT4, SOX2, NANOG, and LIN28. The expression of these sets of transcription factors induced human somatic cells to acquire ES cell-specific characteristics, including morphology, proliferation, and gene- and surface marker expression. Somatic cells reprogrammed in this manner are referred to as induced pluripotent stem (iPS) cells. The existence of iPS cells circumvents the need for blastocysts and reduces concerns associated with immune rejection.

More recently, iPS cells have been generated from a number of different human and murine somatic cell types, such as epithelial, fibroblast, liver, stomach, neural, and pancreatic cells. Further, iPS cells have been successfully differentiated into cells of various lineages (e.g., Dimos et al. Science 321:1218-1221 (2008)).

Current methods for generating iPS cells employ retroviral vectors such as those derived from lentivirus. These vectors stably integrate into, and permanently change, a target cell's DNA at virtually any chromosomal locus. This untargeted interaction between reprogramming vector and genome is associated with a risk of aberrant cellular gene expression as well as neoplastic growth caused by viral gene reactivation (Okita et al. Nature 448:313-317 (2007)). Moreover, continued presence and expression of the transgenes can interfere with the recipient cell's physiology. Further, ectopic expression of transcription factors used to reprogram somatic cells, such as c-Myc, can induce programmed cell death (apoptosis) (Askew et al., Oncogene 6:19151922 (1991), Evan et al., Cell 69:119-128 (1992)). Furthermore, continued expression of factors such as OCT4 can interfere with subsequent differentiation of iPS cells.

Therefore, there is still a need in the art to reprogram somatic cells to a state of higher potency without altering the cells' genetic makeup beyond erasing the epigenetics associated with cell differentiation or pathology. Additionally, there is a need for improved methods to generate a large amount of stem cells that would not be rejected by the recipient. The present disclosure addresses many of the needs mentioned above as well as other objectives that will be appreciated by those skilled in the art.

### SUMMARY

Compositions and methods are provided for the epigenetic erasure and conditioning of cells, particularly animal cells, to a germline, pre-embryonic and highly potent state, which process may be referred to herein as a Janus protocol, the conditioning protocol or as simply the protocol. In some embodiments the conditioned cells, which may be referred to herein as Epinul cells, are totipotent, or cunctipotent. In some embodiments the Epinul cells are derived from somatic mammalian cells. In some embodiments the Epinul cells are generated from an individual of interest for treatment, where the cells are then autologous in relation to the individual. In some embodiments the Epinul cells can differentiate into any somatic or germline cell of the mammal from which the cells are derived. In one embodiment, the stem cells of the present disclosure can reconstitute a whole organism.

In one embodiment, the sample comprising somatic cells is from a mammal. In another embodiment, the mammal is a human. In some embodiments, the sample comprising somatic cells is from one or more organs. In other embodiments, the sample comprising somatic cells is from one or more tissues. In other embodiments the somatic cells are non-mammalian, including without limitation any plant, animal or microbial cell, e.g. plant cells, insect cells, bacterial cells, protozoan cells, and the like.

In one embodiment, methods are provided for generating Epinul cells. The method may utilize the following steps, generally in the order listed, of (a) subjecting a population of cells, e.g. somatic cells, to partial protease digestion, e.g. with trypsin, etc.; (b) suspending the cells in media comprising one or more optional excipients; (c) exposing the cell suspension to environmental pressures sufficient to erase epigenetic programming; and (d) transferring the cells to growth media. The optional excipients may include, for example and without limitation, one or more of epidermal growth factor (EGF); ATP, insulin transferrin selenium (ITS), retinoic acid, ascorbic acid.

The environmental pressures to which the cells are subjected are designed to create stress in the cell, such that molecules involved in epigenetic programming, e.g. methylation of histone proteins, are altered to a state commensurate with that of a germline, pre-embryonic cell. The environmental pressures then induce cellular responses to initiate pro-survival mechanisms. Environmental pressures useful in the methods include, without limitation, cavitation, application of acoustic waves (ultrasound), heat shock, cold shock, application of shear force, application of atmospheric pressure changes, application of fluid viscosity changes, and CO₂ saturation.

In some embodiments, a sequence of cavitation, which may be provided by ultrasound, high temperature, low temperature and protease digestion is applied to the cells, which sequence may be performed in the order indicated, or in a different order; and steps may be repeated, e.g. following treatment of protease, or following cold shock and/or heat shock, cavitation may be additionally applied.

Following the conditioning protocol, the resulting Epinul cells can be grown in culture for a variety of purposes and outcomes. The cells in culture can be directed to a differentiation pathway or pathways by the addition of factors and selection of media and growth conditions. In one embodiment, the Epinul cells differentiate to a blastocyst or morula state, from which pluripotent embryonic stem cells can be derived. In other embodiments the Epinul cells in culture are maintained in a pre-embryonic state.

In some embodiments the highly potent cells Epinul cells are induced to differentiate into somatic cells of interest, e.g. for therapeutic and research interests. Differentiation may proceed through multipotent cell types, e.g., neural stem cells, cardiac stem cells, or hepatic stem cells; or may proceed directly to a more terminally differentiated cell. Differentiated lineages may include, for example and without limitation, any differentiated cells from ectodermal (e.g., neurons and fibroblasts), mesodermal (e.g., cardiomyocytes), or endodermal (e.g., endodermal cells, pancreatic cells) lineages. Specific differentiated cells include, without limitation, pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, endodermal cells, cardiomyocytes, etc.

In some embodiments, methods are provided for regenerative therapy of an individual, e.g. the treatment of prevention of a disease or disorder in a subject in need thereof. Such methods may comprise obtaining somatic cells from the subject in need thereof conditioning the somatic cells in vitro to become Epinul cells; expanding the Epinul cells; differentiating the Epinul cells to the desired multipotent or terminally differentiated cell type; and administering the differentiated cells to the individual. Alternatively the expanded Epinul cells are administered to the subject in need thereof, wherein the Epinul cells generate differentiated cells in vivo that assemble into one or more new tissues or organs following the administration. The methods result in treating or preventing a disease or disorder in a subject in need thereof. The Epinul cells may be autologous with respect to the subject.

In another therapeutic embodiment, methods are provided for repairing and/or regenerating one or more damaged tissues or organs in a subject in need thereof comprising: obtaining somatic cells from the subject in need thereof; conditioning the somatic cells in vitro to become Epinul cells; expanding the Epinul cells; differentiating the Epinul cells to the desired multipotent or terminally differentiated cell type; and administering the differentiated cells to the individual. Alternatively the expanded Epinul cells are administered to the subject in need thereof, wherein the Epinul cells generate differentiated cells in vivo that assemble into one or more new tissues or organs following the administration. The methods result in repairing and/or regenerating the one or more damaged tissues or organs. The Epinul cells may be autologous with respect to the subject.

In another embodiment, a composition is provided, comprising an isolated population of Epinul cells made by the methods or protocols disclosed herein. The cell population may be substantially homogenous, e.g. where at least about 50% of the cells in the population are of the desired phenotype, at least about 75%, at least about 90%, at least about 95% or more. The cells may be in the initial, pre-embryonic, germline state; or may be differentiated an embryonic stem cell state. Alternatively, the cells are differentiated to a multipotent, or terminally differentiated cell. The cell composition may be provided as a pharmaceutical composition, e.g. in a unit dose for administration, in a pharmaceutically acceptable excipient, and the like. Such cell compositions may be provided for use in a method of treating or preventing a disease or disorder in a subject in need thereof. For such purposes, the cells may be autologous with respect to the subject.

In another embodiment, a system for preparing Epinul cells is provided, using the methods of the protocol disclosed herein. In one embodiment, the system is automatable and software configurable, where a device may be provided for manipulation and culture of the cells. In another embodiment, the system comprises a controlled environment. In some embodiments, the system comprises chambers. In some embodiments, the system comprises means to deliver and control environmental pressures. In further embodiments, the system comprises means to grow and maintain cells. In yet another embodiment, the system comprises means to treat cells according to the methods and protocols disclosed herein.

The Epinul cells described herein provide advantages relative to conventional stem cells. Epinul cells are inexpensive to produce, provide a high yield of cells in a scalable process, are capable of differentiation, proliferation, and genetic modification (in vivo and ex vivo), function in a physiologic manner (e.g., conduct, produce, secrete, regulate biologic compounds, etc.), exhibit true totipotency or pluripotency, and are amenable for clinical, diagnostic and commercial uses, such as cell/tissue transplantation, replacement, implantation, grafting, genetic or diagnostic screenings, product development, and for therapeutic, preventative or other treatments purposes (e.g., for spinal cord injuries, other tissue or organ injuries, burns, cirrhosis, hepatitis, Parkinson's Disease, Alzheimer's Disease, stroke, muscular dystrophy, diabetes, arthritis, osteoporosis, leukemia, sickle cell disease and other anemias, as examples). In addition, the present disclosure is well suited for use in individuals who may be sensitive to other treatment options such as persons with cancer or those at a late-stage in their disease, an option not available when using most other traditional methods of stem cell retrieval, such as bone marrow harvest which may only be performed in patients considered relatively healthy. Moreover, the present disclosure is safer than current traditional methods such as bone marrow harvest with no added complications, and may be used repeatedly even on the same patient without any known difficulties or side effects.

The methods of the invention are also advantageous with respect to transplant rejection, as autologous cells can be readily generated for therapy. The present disclosure provides an economic clinical treatment option and fulfills the current need of researchers, healthcare providers, and industry for compositions and method of recruiting and retrieving a large number of stem cells for clinical procedures, including transplantation, gene, protein and cell therapy. The compositions and methods of the present disclosure serve as unique and powerful tools to supply stem cells, especially to a person in need thereof. Custom designed products of the present disclosure include compositions for use in medicinal, therapeutic, diagnostic, engineering, and biotechnology applications, as examples.

The present disclosure provides methods and protocols for the preparation of stem cells (Epinuls) that are pre-embryonic and totipotent, a combination of characteristics that is not possessed by embryonic stem cells (ESCs), induced pluripotent stem cells (iPSCs), or adult tissue-specific stem cells produced by current technologies. The Epinul cells are further capable of forming blastocysts, the inner cell mass of which yield pluripotent, embryonic stem cells.

Those skilled in the art will further appreciate the above-mentioned advantages and superior features of the disclosure, together with other important aspects thereof upon reading the detailed description that follows in conjunction with the drawings.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1H. FIG. 1A Mouse fibroblasts grown in T75 culture flasks are the starting cell colony for this first protocol. These fibroblasts were provided by PrimCell and contain a special GFP cassette that has been inserted into the promoter region of the OCT4 gene. This IRES OCT4 GFP MEF acts as a positive marker and importantly, as a live validation of the core pluripotency marker Pou5f1. This marker will only produce green fluorescing proteins if the gene is active and expressing, otherwise it remains dormant. FIG. 1B Control image showing that there is no fluorescing from the fibroblasts prior to being conditioned with the protocol. The IRES GFP insert will only activate if the core pluripotency genes are activated within the cell. This allows for live visualization of cells that have achieved a state of pluripotency and a way to visually decipher cells that are not expressing genes and remain differentiated or not within the pluripotent classification. FIG. 1C Cell population at 18-24 hours post protocol. IRES OCT4 GFP MEFs are being used to show nuclear expression of OCT4 in live cells. FIG. 1D Positive OCT4 expression shown in cell population (24 hours) post protocol. Quantification of expression documented within first hour post protocol (data not shown here). FIG. 1E Cells are sorted at 24-36 hours by size, utilizing simple 100, 70 and 40 μm mesh screens. FIG. 1F shows early pluripotent stem cell colonies with live OCT4 expression. This is one of the four initial cell populations generated via the protocol. FIG. 1G Stem cell colonies 3 days post protocol. FIG. 1H Shows live OCT4 GFP expression from the expanding colonies.

FIG. 2A-2F. FIG. 2A shows immunofluorescence microscopy of conditioned human stem cells stained for OCT3/4 and counterstained with DAPI. FIG. 2B shows immunofluorescence microscopy of conditioned human stem cells stained for TRA1-60. FIG. 2C shows immunofluorescence microscopy of conditioned human stem cells stained for TRA1-81. FIG. 2D shows immunofluorescence microscopy of conditioned mouse cells stained for SSEA-1. FIG. 2E shows immunofluorescence microscopy of conditioned mouse cells stained for OCT3/4. FIG. 2F shows immunofluorescence microscopy of conditioned mouse cells stained for SOX2.

FIG. 3 shows RT-PCR on six cell samples that have been treated according to the protocol of the present disclosure to measure expression of stem cell marker genes. Testing was performed 36 hours post protocol. The control in lane 7 is MRHF cells that have not undergone the protocol.

FIG. 4A-4J. Germinal cells developing over the course of 5 days. These are a typical outcome of the protocol, with approximately 40% of initial “intake” cells being converted to embryos. 80% or more of these will progress through normal development and hatch from the blastocyst. Culturing these hatched cells produces a robust and highly self-replicating embryonic-like stem cell colony.

FIG. 5A-5C shows stem cells derived from the inner cell mass of blastocysts produced from the protocol of the present disclosure. FIG. 5A, bright field microscope image of cells; FIG. 5B, cells stained for Oct4; FIG. 5C, cells stained with DAPI.

FIG. 6A-6B shows pluripotent embryonic stem cells from blastocysts produced by the protocol of the present disclosure stained with DAPI FIG. 6A and stained for Nanog FIG. 6B.

FIG. 7A-7C shows bright field microscope image of embryonic stem cells from mouse cells FIG. 7A and stained with DAPI FIG. 7C. The stem cells were derived from the inner cell mass of blastocysts produced from the protocol of the present disclosure FIG. 7B.

FIG. 8A-8E. Allowed the cells to differentiate through growth factors added to media, the neurons produced remained in normal morphological state, produced new dendrites and interconnected with neighboring cells. Upon further splitting and passaging, new cell colonies continued to maintain a normal growth pattern. No signs of mutagenesis, tumor formations or clumping, as well as well-defined networks. FIG. 8A Janus transformed human cells were cultured as one-day-old EB aggregates with BMP4 for 4 days in in the presence and absence of 5 ng/ml BMP4 FIG. 8B-8E after 4 days of culturing, neurons produced with new dendrites and interconnections with neighboring cells

FIG. 9A-9N. Differentiation of cardiac cells. FIG. 9A. Embryoid bodies forming after removal of stem cell factors from media. FIG. 9B-9E Derived cardiac cells growing on a gelatin basement membrane in 6 well plates. FIG. 9F-9J Cardiomyocytes as well as other cardiac cells are prepped for derivation 24 hours post protocol and early populations are seen within 4-5 days. FIG. 9K and FIG. 9M Brightfield microscopy of cardiomyocyte characteristic cardiomyocyte structures are visible. FIG. 9L & FIG. 9N Sarcomeric alpha Actin/Actinin, a cardiomyocyte-specific marker was used for immunofluorescence staining of methanol-fixed directly derived cells—Intercalated discs, nuclei, and banding are visible. Direct differentiation protocols allow for cells produced from the Janus protocol to be directly differentiated into differentiated cells. Cardiac cells are exemplified here. For immunostaining (FIGS. 9L & N), Cells were fixed either with 4% paraformaldehyde solution in phosphate-buffered saline (PBS), permeabilized with 0.1% Triton-X100 in PBS for 20 minutes, blocking solution of 10% normal goat serum, or alternately fixed and permeabilized with −20 C, methanol for 10 mins, followed by monoclonal antibodies to sarcomeric α-actinin (1A4) conjugated Alexa Fluor® 488 (Santa Cruz Biotechnology, Inc.) in 1:200 dilution for 30 minutes.

FIG. 10 provides a schematic of the epigenetic erasure conditioning protocol, and the cells that are generated by the protocol.

FIG. 11 provides a schematic of a system for conditioning cells according to the methods described herein.

### DETAILED DESCRIPTION

Although making and using various embodiments of the present disclosure are discussed in detail below, it should be appreciated that the present disclosure provides many inventive concepts that may be embodied in a wide variety of contexts. The specific aspects and embodiments discussed herein are merely illustrative of ways to make and use the disclosure, and do not limit the scope of the disclosure.

### Definitions

Unless defined otherwise, all technical and scientific terms used herein have the same meaning as commonly understood by those of ordinary skill in the art to which the disclosure belongs. For the purposes of the present disclosure, the following terms are defined below. The articles “a,” “an,” and “the” are used herein to refer to one or to more than one (i.e. to at least one) of the grammatical object of the article. By way of example, “a cell” means one cell or more than one cell.

As used herein, the term “comprising” or “comprises” is used in reference to compositions, methods, and respective component(s) thereof, that are essential to the disclosure, yet open to the inclusion of unspecified elements, whether essential or not.

As used herein, the term “consisting of” refers to compositions, methods, and respective components thereof as described herein, which are exclusive of any element not recited in that description of the embodiment.

As used herein, the term “consisting essentially of” includes any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-5698). Further, unless otherwise required by context, singular terms shall include pluralities and plural terms shall include the singular.

Unless otherwise stated, the present disclosure was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005) and Animal Cell Culture Methods (Methods in Cell Biology, Vol. 57, Jennie P. Mather and David Barnes editors, Academic Press, 1st edition, 1998) which are all herein incorporated by reference in their entireties.

As used herein, “assemble” refers to the assembly of differentiated cells generated from somatic stem cells into functional organ structures i.e., myocardium and/or myocardial cells, arteries, arterioles, capillaries, kidney tubules, alveolar epithelium, intestinal epithelial villus/crypt structures, etc.

As used herein, “autologous” refers to cells or tissues obtained from the same individual. In the context of stem cell therapy, the donor and recipient are the same person. In one embodiment, the stem cells of the present disclosure are prepared from a given individual and are used, for example, in stem cell therapy in the same individual.

### Epigenetic Erasure and Epinul Definitions

As used herein, “Epinul” refers to the engineered, highly potent stem cells with epigenetic signals consistent with primordial cells produced using the methods of epigenetic erasure described herein, e.g. removal of methylation. Epinul cells are a fertile, epigenetically clean and highly potent cell type. In some embodiments Epinul stem cells are totipotent. In some embodiments a population of genetically and epigenetically identical Epinul cells are provided. For transplantation purposes, a population of Epinul cells can be produced that is autologous to the intended transplant recipient.

As used herein, “Epinul Cells” refer to the highly potent stem cells produced by the methods of the present disclosure. In some embodiments the Epinul stem cells are cunctipotent or totipotent, and have the capacity to give rise to any of the somatic or germline cells of the animal from which the Epinul cell is derived. Derivatives of these cells and other produced product may carry the “c” conditioned prefix, followed by the cell or tissue type and its potency or lineage. Phenotypically, Epinul can be very small, round cells. The most highly potent cells may be referred to as cunctipotent Epinul cells, or cEpinul. The cells are generated in the absence of: introducing exogenous genes into the cell, including without limitation transcription factors, exogenous mRNA transcripts; exogenous transcription factors into the cell; and in the absence of nuclear fusion.

As used herein, “Basic Biological Nuclear Preservation” or “BBNP” refers to the process of remodeling and preparing the histone and chromatin of the nuclear DNA to enact a holistic preservation reaction.

Bivalent chromatin, as used herein, refers to regions of DNA that are bound to histones, and which have both repressing and activating transcriptional regulators in the same region. In bivalent chromatin, both types of regulators are interacting with the same DNA sequence at the same time. These domains are involved in developmental regulation in highly potent cells. Examples of antagonistic epigenetic regulators include methylation at specific positions of histones. Bivalent chromatin domains are found in germline and embryonic stem cells.

“Epigenetic erasure” refers to the process described herein to reset the developmental potential of the cell, such that the cell can then be readily differentiated to a desired cell type. This process sheds the epigenetic modification of DNA by methylation and the association of DNA with chromatin and histones, which allows the cell totipotential growth and differentiation. As such, the biological clock of these structures is reset, and a reversion to germ line, pre-embryonic state is achieved.

As used herein, a “starting cell population”, or “initial cell population” refers to a somatic cell, usually a primary, or non-transformed, somatic cell, which undergoes epigenetic erasure by the methods described herein. Sources of starting cell populations include individuals desirous of cellular therapy, individuals having a genetic defect of interest for study, and the like. In some embodiments, cells obtained from a subject for the purpose of epigenetic erasure are chosen from any animal cell type, including fibroblast cells, adipose tissue cells, mesenchymal cells, bone marrow cells, stomach cells, liver cells, epithelial cells, nasal epithelial cells, mucosal epithelial cells, follicular cells, connective tissue cells, muscle cells, bone cells, cartilage cells, gastrointestinal cells, splenic cells, kidney cells, lung cells, testicular cells, nervous tissue cells, etc. In some embodiments, the cell type is a fibroblast, which may be conveniently obtained from a subject by a punch biopsy. In certain embodiments, the cells are obtained from subjects known or suspected to have a copy number variation (CNV) or mutation of the gene of interest. In other embodiments, the cells are from a patient presenting with idiopathic/sporadic form of a disease. The cells are then conditioned, and may be transdifferentiated to adopt a specific cell fate, such as endodermal cells, neuronal cells, for example dopaminergic, cholinergic, serotonergic, GABAergic, or glutamatergic neuronal cell; pancreatic cells, e.g. islet cells, muscle cells including without limitation cardiomyocytes, hematopoietic cells, and the like.

The term “efficiency of conditioning” may be used to refer to the ability of cells to give rise to conditioned cells. The term “efficiency of conditioning” may also refer to the ability of somatic cells to be differentiated to a substantially different somatic cell type. The term “efficiency of reprogramming” may also refer to the extent of telomere extension when conditioning a cell from a senescent to a juvenile form. The efficiency will vary with the particular combination of somatic cells, method of epigenetic erasure, and method of culture following induction of reprogramming.

### Stem Cell Definitions

In general, a stem cell is a multipotent or pluripotent cell in an unspecialized (e.g., undifferentiated) state that may give rise to one or more unspecialized cells. One critical identifying feature of a stem cell is its ability to exhibit self-renewal or to generate more of itself; therefore, a cell with the capacity for self-maintenance. In addition, as used herein, a stem cell is an unspecialized cell capable of proliferation (replication many times over), self-maintenance, and production of a large number of specialized functional progeny, as well as an ability to regenerate tissue after injury. Various specific stem cells are defined below.

Pre-Germinal Progenitor/Primary Cunctipotent cells are derived by the methods described herein. These small cells, from about 1-4 μm in diameter are universal progenitor cell giving rise to sex cells, primordial germ cells, pre-stage of germinal stem cells, oogonia, oocytes and male germ cells, as well as the template cell for the totipotent blastomeres that make up the early stage morula.

Primordial Germ Cells (PGC) are any of the large spheric diploid cells that are formed in the early stages of embryonic development and are precursors of the oogonia and spermatogonia. They are formed outside the gonads and migrate to the embryonic ovaries and testes for maturation.

Germinal Cells. A germ cell is any biological cell that gives rise to the gametes of an organism that reproduces sexually.

Fully competent Clonal Stem Cells are the replacement lines of what are conventionally referred to as embryonic stem cells (ESC) and iPSC

Epi-blast (inner cell mass derived) embryonic stem cells are stem cell colonies produced from the removal and plating of inner cell mass from synthetic engineered blastocyst.

As used herein, the term “embryonic stem cells” refers to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). A cell has the phenotype of an embryonic stem cell if it possesses one or more of the unique characteristics of an embryonic stem cell which include, without limitation, gene expression profile, proliferative capacity, differentiation capacity, karyotype, responsiveness to particular culture conditions, and the like. Embryonic stem cells are a man-made construct, created by harvesting pluripotent cells, which exist only at the earliest stages of embryonic development as a single cell source from a blastocyst. In humans, these cells no longer exist after about five days of development. Importantly and by lack of developmental instructions from surrounding cell structures, there are numerous genes from each of the other cell layers in the blastocyst that are not expressed in embryonic/iPSC stem cells. While they are classified as “pluripotent” capable of producing more than 200 different cell types in the body, it has been demonstrated over and over for decades that the cells or tissues differentiated from these starting stem cells lack functional properties required to allow them to perform within an organism.

As used herein, the term “induced pluripotent stem cells” or, iPSCs, refers to cells that have been reprogrammed to pluripotency by inducing expression of specific “reprogramming factors”, which are generally produced from differentiated adult, neonatal or fetal cells. The iPSCs produced do not refer to cells as they are found in nature.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “adult stem cell”, or “somatic stem cell” refers to a multipotent stem cell derived from non-embryonic tissue, including fetal, juvenile, and adult tissue. Somatic stem cells can be of non-fetal origin. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

As used herein, the term “totipotent” refers to a cell that has the potential to develop into any cell found in a body, such as a body of a mammal. As one example, a totipotent zygote cell is created when a single celled sperm and egg unite. This totipotent fertilized egg has the potential to give rise to virtually all human cells, such as nerve or heart. It is during the early cell divisions in embryonic development that more totipotent cells are produced. Within several days, these totipotent cells divide and create replicas, therefore producing more totipotent cells. It is after approximately four days that the cells begin to specialize into pluripotent cells, which can go on to specialize further but can't ever produce an entire organism as totipotent cells can.

As used herein, the term “pluripotent” refers to a cell with the capacity, under different conditions, to commit to one or more specific cell type lineage and differentiate to more than one differentiated cell type of the committed lineage, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the term “progenitor” cell refers to cells that have a cellular phenotype that is more primitive (i.e., is at an earlier step along a developmental pathway or progression than is a fully differentiated or terminally differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Progenitor cells give rise to precursor cells of specific determinate lineage, for example, certain lung progenitor cells divide to give pulmonary epithelial lineage precursor cells. These precursor cells divide and give rise to many cells that terminally differentiate to pulmonary epithelial cells.

As used herein, the term “precursor” cell refers to cells that have a cellular phenotype that is more primitive than a terminally differentiated cell but is less primitive than a stem cell or progenitor cells that is along its same developmental pathway. A “precursor” cell is often a progeny cell of a “progenitor” cell which are some of the daughter of “stem cells”. One of the daughters in a typical asymmetrical cell division assumes the role of the stem cell.

As used herein, the terms “renewal” or “self-renewal” or “proliferation” are used interchangeably herein and refer to the ability of stem cells to renew themselves by dividing into the same non-specialized cell type over long periods, and/or many months to years. A feature of somatic stem cells is asymmetric replication, where after cell division one daughter cell maintains the stem cell phenotype, and the other daughter cell differentiates.

### Differentiated Cell Definitions

As used herein, in the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a lung stem cell), which in turn can differentiate into other types of precursor cells further down the pathway (such as a thymocyte, or a T lymphocyte precursor), and then to an end-stage differentiated cell, which plays a characteristic role in a certain tissue type, and may or may not retain the capacity to proliferate further.

As used herein, the term “differentiated cell” refers to any primary cell that is not, in its native form, pluripotent as that term is defined herein. Stated another way, the term “differentiated cell” refers to a cell of a more specialized cell type derived from a cell of a less specialized cell type (e.g., a stem cell such as a lung stem cell) in a cellular differentiation process. For example, a pluripotent stem cell in the course of normal ontogeny can differentiate to the mesodermal layer, from which forms hematopoietic stem cells, which are maintained as somatic stem cells throughout adult life. These somatic stem cells give rise to all differentiated blood cells.

As used herein, the term “transdifferentiation” refers to lineage reprogramming, a process where a somatic cell type is transformed into a different somatic cell type without proceeding through an intermediate pluripotent state or progenitor cell type. Transdifferentiation may also refer to cell fate switches, including the interconversion of stem cells from one type to a different type. Current uses of transdifferentiation include disease modeling and drug discovery and in the future may include gene therapy and regenerative medicine.

As used herein, the term “somatic cell” refers to any cells forming the body of an organism other than germline cells. Germline cells include primordial germ cells (PGC), which are set aside in early embryogenesis, and the cells that PGC differentiate in to, i.e. sperm or egg, also known as “gametes”. Every non-germline cell in the body is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

As used herein, the term “adult cell” refers to a cell found throughout the body after embryonic development.

As used herein, “in vivo” refers to those methods using a whole, living organism, such as a human subject. As used herein, “ex vivo” refers to those methods that are performed outside the body of a subject, and refers to those procedures in which an organ, cells, or tissue are taken from a living subject for a procedure, e.g., isolating cells from a tissue obtained from a donor subject, preparing Epinul and then administering the isolated Epinul to a recipient subject. As used herein, “in vitro” refers to those methods performed outside of a subject, such as an in vitro cell culture experiment. For example, Epinuls can be cultured in vitro to expand or increase the number of Epinuls, or to direct differentiation of the Epinuls to a specific lineage or cell type, e.g., respiratory epithelial cells, prior to being used or administered according to the methods described herein.

As used herein, the term “phenotype” refers to one or a number of total biological characteristics that define the cell or organism under a particular set of environmental conditions and factors, regardless of the actual genotype, for example, the expression of cell surface markers in a cell.

### Cell Culture Definitions

As used herein, the term “cell culture medium” (also referred to herein as a “culture medium” or “medium” or “media”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. The cell culture medium can be a chemically defined animal (xeno)-free and serum-free medium. Cell culture media ordinarily used for particular cell types are known to those skilled in the art.

When culturing stem cells, the culture medium is generally one used under standard conditions appropriate for stem cells or for cell specialization. Supplementation may occur with a growth-promoting signal or substituent, as known to one of ordinary skill in the art. As used herein, the terms “growth-promoting signal,” “growth-promoting substituent,” “proliferation-inducing signal,” “proliferation-inducing substituent,” or “growth factor” generally refer to a compound (e.g., protein, peptide or other molecule) or stimulus having a growth, proliferative, and/or trophic effect on a cell.

As used herein, “differentiation-inducing signal,” “differentiation-inducing substituent,” generally refers to a compound or stimulus that induces cell differentiation or specialization. Such compounds may be referred to generally as “signals” or “substituents.” Those of ordinary skill in the art will recognize that one or more substituents may be used in combination and combinations that promote growth, proliferation, and cell-type specific differentiation are known in the art, thus inducing growth, proliferation, and differentiation does not require undue experimentation. Examples of signals or substituents include cytokines, growth-promoting pharmaceutical agents, growth factors such as epidermal growth factor (EGF), amphiregulin, fibroblast growth factor (FGF, acidic or basic), nerve growth factor (NGF), platelet-derived growth factor (PDGF), thyrotropin releasing hormone (TRH), transforming growth factor (TGF), and insulin-like growth factor (IGF), as examples. Such substituents may be added to the culture medium at concentrations ranging between about 1 fg/ml to 1 mg/ml. To optimize culture conditions, simple titrations can be performed easily to determine optimal substituent concentrations. Additional signals include mechanical and/or electrical signals (e.g., cell-cell contact, adhesion, movement, electrical stimulation, physical pressure, distortion, etc.).

Other substituents may include, without limitation, VPA 0.5-2 mM; SAHA 5 μM; TSA 20 nM; Sodium butyrate 0.5-1 mM; 5-aza-CR, AZA 0.5 mM; Tranylcypromine (Parnate) 5-10 μM; DZNep 0.05-0.1 μM; TTNPB 1 μM; SB431542 10 μM; PD0325901 1 μM; Y27632 10 μM; GSK-3β inhibitor; LSD1 inhibitor LiCI 5-10 mM; A83-01 0.5 μM; Rapamycin 0.3 nM; IP3K inhibitor 12 μM; P38 kinase inhibitor 1-2 μM; DNP 1 μM; BIX 0.5-2 μM; CHIR 3-10 μM; 616452 (E616452, Repsox) 1 μM; Repsox (616452) 5-10 μM; Forskolin 20-50 μM; Chir99021 10 μM; VPA 0.5 mM; PD0325901 1 μM; Chir99021 10 μM; CYT296 (125 nM); Forskolin—A 50 mM (5 mg/244 μl) in DMSO; trans-Retinoic Acid—Solubility DMSO (10 ng/ml); ATP 10 mM; L-Ascorbic Acid (50 ng/ml); Flt3 ligand (10 ng/ml); EGF at 1-10 10 μg/ml; ATP; ITS 0.5 ml/50 ml.

As used herein, the term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

As used herein, the term “isolated population” with respect to an isolated population of cells as used herein refers to a population of cells that has been removed and separated from a mixed or heterogeneous population of cells. In some embodiments, an isolated population is a substantially homogenous population of cells as compared to the heterogeneous population from which the cells were isolated or enriched from, e.g. where the cells in the population are at least about 50% a single cell type, at least about 75%, at least about 90%, at least about 95% or more.

As used herein, a “cell line” is a population of cells that can be propagated in culture through at least 10 passages. The population can be phenotypically homogeneous, or the population can be a mixture of measurably different phenotypes. Characteristics of the cell line are those characteristics of the population as a whole that are essentially unaltered after 10 passages.

### Additional Definitions

As used herein “damaged tissue” refers to tissue or cells of an organ which have been exposed to ischemic or toxic conditions that cause the cells in the exposed tissue to die. Ischemic conditions may be caused, for example, by a lack of blood flow due to stroke, aneurysm, myocardial infarction, or other cardiovascular disease or related complaint. The lack of oxygen causes the death of the cells in the surrounding area, leaving an infarct, which will eventually scar. Ischemia may occur in any organ that is suffering a lack of oxygen supply.

As used herein, the term “tissue” refers to a group or layer of specialized cells, which together perform certain special functions. The term “tissue-specific” refers to a source of cells from a specific tissue.

As used herein, the term “organ” refers to two or more adjacent layers of tissue, which layers of tissue maintain some form of cell-cell and/or cell-matrix interaction to form a microarchitecture.

As used herein, the term “genetically altered” or “transformed” refers to a cell where a polynucleotide has been transferred into the cell by any suitable means of artificial manipulation, or where the cell is a progeny of the originally altered cell that has inherited the polynucleotide. The polynucleotide may contain a sequence that is exogenous to the cell, it may contain native sequences in an artificial arrangement (e.g., an encoding region linked to a different promoter), or it may provide additional copies of a native encoding sequence. Unless explicitly stated otherwise, the process of transferring the polynucleotide into the cell can be achieved by any technique suitable for the application at hand, which may include but is not limited to electroporation or liposome-mediated transfer, homologous recombination, transduction or transfection using a viral or bacterial vector. The polynucleotide will often comprise a transcribable sequence encoding a protein of interest, which enables the cell to express the protein at an elevated level. Also included are genetic alterations by any means that result in functionally altering or abolishing the action of an endogenous gene. Suitable methods for effecting such alterations include homologous recombination using a suitable targeting vector (U.S. Pat. Nos. 5,464,764, 5,631,153, 5,789,215, 5,589,369 and 5,776,774, each of which is incorporated herein in its entirety).

The genetic alteration is said to be “inheritable” if progeny of the altered cell has the same alteration. Determination of whether the genetic alteration is inheritable can be made by detecting presence of the polynucleotide template (e.g., by PCR amplification), or by detecting a phenotypic feature (such as expression of a gene product or effect thereof) that depends on the genetic alteration to be manifest.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, a “vector” refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondria! DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” in intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

As used herein, a “therapeutically sufficient amount” or “therapeutically effective amount” includes within its meaning a non-toxic but sufficient and/or effective amount of the particular therapeutic and/or pharmaceutical composition, e.g. a regenerative cell composition, to provide a desired therapeutic effect. The exact amount required will vary from subject to subject depending on factors such as the patient's general health, the patient's age and the stage and severity of the condition. In particular embodiments, a therapeutically sufficient amount is sufficient and/or effective to ameliorate, reduce, and/or improve at least one symptom associated with a disease or condition of the subject being treated.

As used herein, the term “transplantation” refers to a process where a cell, a tissue or an organ is removed from a donor or otherwise prepared ex vivo, e.g. using cell culture, cell isolation techniques, genetic engineering, and the like; and implanted into the recipient. The recipient may receive a cell, a tissue or an organ from an MHC-matched donor (allogeneic transplantation) or from autologous cells subjected to ex vivo treatment. When allogeneic or xenogeneic cells are used, rejection responses may optionally obviated by any method known in the art such as administering one or more immunosuppressive agents (e.g. azathiopurine, cyclophosphamide etc.).

As used herein, “patient” or “subject” may encompass any vertebrate including but not limited to humans, mammals, reptiles, amphibians and fish. The patient or subject is frequently a mammal such as a human, or a domesticated mammal, e.g., dog, cat, horse, and the like, or production mammal, e.g., cow, sheep, pig, and the like. In one embodiment, the patient is a human patient.

### Methods of Epigenetic Conditioning and Erasure (Janus Protocol)

The present disclosure provides stem cells that are able to differentiate into non-derived tissue (e.g., a cell type that differs from the tissue from which they were derived). This ability is termed plasticity. Thus, stem cells of the present disclosure are able to function by (a) self-renewal and (b) differentiation into a number of specialized cell types. Using the protocol of the present disclosure, conditioned stem cells are generated from a host sample of adult cells and/or tissues; when stem cells have the phenotype of a germline, pre-embryonic stage cell (Epinul). In some embodiments these cells are totipotent, and can be maintained by self-renewal, or can be differentiated into cells to produce blastocysts, embryonic-like stem cells produced from the inner cell mass via various methods, direct production of genetically identical stem cells, embryoid bodies and lower order progenitor stem cells. Such totipotent cells can also be directly differentiated into cell types in all three germ layers. The Epinul of the present disclosure can be distinguished from conventionally cultured stem cells, such as embryonic stem cells (ESCs), induced pluripotent stem cells (iPSCs) and adult tissue-specific stem cells by virtue of the pre-embryonic, germline stem cell phenotype. A feature of the methods is the ability to rapidly generate highly potent cells from an individual, for example for use in autologous regenerative therapies. This combination of characteristics is not possessed by ESCs, iPSCs or adult tissue-specific stem cells produced by current technologies.

The protocol of the present disclosure allows the production of self-similar pre-embryonic cell products created from a specific individual that inherit the specific and unique genetic map of that individual. The present disclosure provides a platform to produce completely potent and viable stem cells that are genetically compatible with a given individual or recipient.

In one embodiment, Epinul cells are produced by mechanistic, chemical and/or electromagnetic means. Epinul cells have a characteristic round, almost bubbly, shiny appearance, with a majority of the cell consisting of nuclei surrounded by a thin rim of cytoplasm. They spontaneously differentiate into viable oocyte, zygote and morula-like structures, embryoid body structures, and pluripotent embryonic stem cell colonies and can be induced to other cell lineages directly by differential supplementation of protocol and post-protocol media with various factors. Stem colonies spontaneously form from protocol but can also be derived from the morula or blastocyst structures. Epinul cells are positive for nuclear Oct-4, Oct-4A, Nanog, Sox-2, and TERT detected by RT-PCR and immunostaining. Oocyte structures tested positive for germ cell markers, e.g. c-Kit, DAZL, GDF-9, VASA, and ZP4.

In one embodiment, one or more directed environmental pressures are applied to the surrounding cell environment, the cell and to cellular signaling pathways. The environmental pressure applied to somatic cells produces Epinul cells. These cells are fully capable of generating any cell or tissue in the body and represent a therapeutic platform to address current demands in regenerative medicine, research, drug discovery, infertility and many other applications. Epinul cells may be autologous to an individual of interest, may be genetically identical and stable, and importantly are free of any exogenous transcription factors, RNAs or other small molecules, exogenous viral or nuclear manipulations. Further, these cells contain the mitochondria! DNA from the host, unlike nuclear transfer, so epigenetics across the entire cell and surrounding cellular signaling is self-similar.

The highly potent nature of the Epinul cells allows for the generation of replacement tissues and, organs, and even whole organisms. Further, the cloning capacity of the Epinul cells are useful in animal models, blood banks, consumables and a host of other applications utilizing adult cells, tissues and any other sources of viable, live cells. Methods of the present disclosure do not utilize exogenous transcription factors, RNAs or other small molecules, and do not utilize any form of viral or non-viral insertion of materials into the nuclear DNA of the targeted cells.

Without being bound by the theory, it is believed that the methods of applied environmental pressures force the cells to invoke protective measures to preserve nuclear DNA in a viable form for reproduction/replication. Across species, many beneficial protective and regenerative actions are possible given the proper set of circumstances and the required “raw materials” to perform these feats.

The present disclosure uses a combination of environmental factors, including but not limited to, mechanical, chemical and electromagnetic operations, to stimulate various signaling pathways in the source protocol cells, producing specific responses to that environmental pressure. A synthesis of the various mechanistic protocols combine the cellular responses and released products from these responses into a potent solution of enzymes, proteins, growth factors and transcription factors that are required to fully transdifferentiate, destroy the identity of the former cell and produce new, bivalent chromatin-containing totipotent cells. This open chromatin structure is similar to fetal germ cells in meiotic and post meiotic stages and, in one embodiment, can be the continuity factor and mitotically heritable feature of these totipotent stem cells. Maintenance of the inherited H3K4me3 and H3K27me3 modifications is crucial for a truly autologous process as well as exact differentiation into germ or somatic cells. The variations of these sites in diseased cells may represent a model for detecting and correcting inherited disorders or disease that has mutated cells. Deriving therapeutic solutions for an individual from a Epinul cell, zygote or cESC cell requires the maintenance of the epigenetically clean “blueprint” that was created in the crossover of genetics from the host parents. This blueprint serves as a conditioned environment of open chromatin across a wide range of genes responsible for the ability to specialize and/or derive further somatic lineages and a signaling capacity to the environment that it belongs, but additionally has a higher order function than the somatic derivatives. In the condensing process of mitotic division, chromatin regulatory factors, transcription factors and other epigenetic markers are discarded or enzymatically removed, while preserving the bivalent H3K4me3- and H3K27me3-marked histones and associated methyltransferases. As such, a forced division or perhaps better classified as an emergent division, mimicking passive DNA production and “naked DNA” production loses “epigenetic memory” of former cell fate, but maintains the overall blueprint. It is postulated that this bivalent structure persists throughout the entire system and within the differentiated cells, only in a highly restricted manner, orchestrated according to the lineage specific needs.

There are numerous types of pressures, damage, disruption and stress that can be applied to a cell or that a cell can experience. Depending on the type and level of the insult, a cell can activate varying responses to combat these environmental assaults. As such, a carefully crafted series of external mechanical factors are required to properly initiate a cascade of reactions, in a proper sequence to initialize an environment that enacts a “reboot” for the cell, driving it into an open and pluripotent format, capable of repopulating the effected system by producing newly formed cells/tissues from an embryonic like stage.

In one embodiment of the present disclosure, the methods of a protocol is designed to artificially construct an environment for an in vitro culture of cells to undergo a radical treatment of elemental, environmental, chemical and electrodynamic cues to trigger a specific cascade of events that leads to the destruction of the somatic cell construct. The protocol of the present disclosure varies significantly and completely from the textbook understanding of causal interactions within a cell and of the cell itself.

In some embodiments, the methods deliver precise forms of stimulus, in periodic administrations, accentuated by the introduction of external energy in the form of ATP, amino acids, limited growth factors and manipulation of the media environment of the cell, with three requirements: 1) reconstruct the bivalent CpG landscape of a pre-embryonic totipotent cell, 2) ensure that no genetic modifications or mutations are enacted upon the nuclear DNA or mitochondrial DNA of the cell, and 3) positively verify and optimize construct of the “radio” like messaging system being utilized by the DNA and cell network for communication.

In one embodiment, a protocol includes a set of predetermined changes to the cell media and cells that initiate the Heat/Cold shock family of proteins to act as cell level supervisors and protect mitochondria and DNA throughout the destruction and de novo creation of a temporary cellular membrane, while temporarily arresting the mitotic process and enacting a tightly regulated transcriptional activation of a growing cascade of core transcription factors and associated pathways.

In other embodiments, the protocol initializes the process with manual stress through perturbations of the media, thus causing not only an early heat shock response, but employing these stress factors in a manner that calls upon the recruitment of all of the major HSPs. By creating considerable atmospheric pressure changes, as well as fluid viscosity changes, oxygen saturation, CO₂ saturation, and the creation of shear forces, the cells are temporarily introduced to heavy external pressure, expansion back into a less pressurized environment and back to a stable pressure in a frequency dependent manner via careful and specific methods of trituration through larger and smaller channels.

In some embodiments, the cells are suspended in medium comprising ATP, amino acids, and serum, and may further comprise one or both of Insulin Transferring Selenium (ITS), Epidermal growth factor (EGF). Critically to the ATP addition, there is a rise in intracellular calcium levels. In one embodiment, glycolysis and protein synthesis as well as activation of a number of early and critically important genes is induced, producing specific proteins and transcription factors. Activated genes may include Heparin-binding EGF-like growth factor (HB-EGF), transforming growth factor-α (TGF-α), Amphiregulin (AR), Epiregulin (EPR), Epigen, Betacellulin (BTC) and the neuregulin class of proteins.

In one embodiment of the present disclosure, ATP is added to the media in the initial steps of the protocol. In some embodiments, the models calculated in the present disclosure combine the average expected percentage of ATP expected within a cell population of the size utilized within the protocol, the Gibbs free energy model of that population and then adjusts the proportion according to the calculations based on required energy for the activation, translation and interruption of cascades. This can be as concise as needed, but since the body does not store extra quantities of ATP, an over calculation of requirements is recommended. Typically, the ratio of equilibrium giving the Gibbs free energy is around 10× shift from equilibrium, which produces a large amount of energy when ATP is hydrolyzed. The Energy charge calculations take into account the availability of alkaline metals ions, overall ionic strength and the current population's ability to present Mg2+ and Ca2+ for the reactions. Since the typical reaction is significantly less than even basic requirements for disrupting the CpG bonds and initializing transcription via this method, the base calculation of ΔG, representing −57 kJ/mol (−14 kcal/mol) per available reaction seeding the environment with external ATP, as well as thermal, electrical and energy created from various applied forces. With the addition of a significantly higher equilibrium imbalance, the increase of energy is significantly higher and continuous allowing for immediate access and a temporary excess, distributing the gains as fuel to maintain the massive changes from external influences and the cells innate directives to produce the highest order DNA preservation and proliferation construct possible, in the form of totipotent pre-embryonic cells.

In one embodiment, methods are provided for generating Epinul cells. The method may utilize the following steps, generally in the order listed, of (a) subjecting a population of cells, e.g. somatic cells, to partial protease digestion, e.g. with trypsin, etc.; (b) suspending the cells in media comprising one or more optional excipients; (c) exposing the cell suspension to environmental pressures sufficient to erase epigenetic programming; and (d) transferring the cells to growth media. The optional excipients may include, for example and without limitation, one or more of epidermal growth factor (EGF); ATP, insulin transferrin selenium (ITS), retinoic acid, ascorbic acid.

The environmental pressures to which the cells are subjected are designed to create stress in the cell, such that molecules involved in epigenetic programming, e.g. methylation of histone proteins, are altered to a state commensurate with that of a germline, pre-embryonic cell. The environmental pressures then induce cellular responses to initiate pro-survival mechanisms. Environmental pressures useful in the methods include, without limitation, cavitation, application of acoustic waves, heat shock, cold shock, application of shear force, application of atmospheric pressure changes, application of fluid viscosity changes, and CO₂ saturation.

In some embodiments, a specific sequence of temperature shock and mechanical force is applied to the cells, comprising partial protease digestion, cold shock, ultrasound and pipetting; heat shock, cold shock. Optionally a second proteinase digestion is then used. The steps are typically performed in culture medium, e.g. DMEM.

In one embodiment a population of somatic cells is first contacted with a protease, including without limitation trypsin, for a partial protease digestion. The concentration of protease and period of time is sufficient to release adherent cells. Cells in suspension are contacted with the trypsin for a similar period of time, e.g. up to about 5 minutes, about 10 minutes, about 15 minutes, about 20 minutes, about 30 minutes.

The protease-treated cells are diluted into cold medium to neutralize the protease, e.g. diluting 1:1; 1:2; 1:3; 1:5, etc. The cell concentration following dilution may be from about 1×10⁵ to about 1×10⁷ cells/ml, e.g. about 5×10⁵, 10⁶, 5×10⁶, etc.

The cells are then subjected to mechanical stress, including without limitation ultrasound and pipetting through a small bore, usually where the bore is larger than the cell, e.g. a conventional 1, 5, 10 ml. pipette. A cell can be caused by vacuum pressure and/or the flow of a fluid, to pass through a pipette.

The steps may be performed in medium, for example DMEM, comprising one or more factors selected from ATP, EGF, IGF-I, L-ascorbic acid, retinoic acid, proteinase K. In some embodiments the medium comprises ATP. In some embodiments the medium comprises ATP and EGF. In some embodiments the medium comprises ATP and retinoic acid, optionally with EGF. In some embodiments the medium comprises all the listed factors.

Concentrations of the factors may be as follows: ATP at a concentration of from about 1 μM to about 500 mM, e.g. up to about 5 μM, up to about 10 μM, up to about 25 μM, up to about 50 μM, up to about 100 μM or more. Alternatively ATP can be added after application of environmental pressures.

EGF may be present at a concentration of from about 0.5 ng/ml to about 250 ng/ml, e.g. at least about 0.5 ng/ml, at least about 1 ng/ml, at least about 5 ng/ml, at least about 10 ng/ml, at least about 50 ng/ml, at least about 100 ng/ml or more.

L-ascorbic acid may be present at a concentration of from about 1 ng/ml to about 500 ng/ml, e.g. at least about 1 ng/ml, at least about 5 ng/ml, at least about 10 ng/ml, at least about 50 ng/ml, at least about 100 ng/ml, at least about 500 ng/ml or more.

Retinoic acid may be present at a concentration of at least about 1 nM, at least about 10 nM, at least about 100 nM, up to about up to about 10 μM to about 100 μM up to about 1 mM.

Proteinase K may be present at a concentration of from about 0.5 μg/ml to about 500 μg/ml, e.g. at least about 0.5 μg/ml, at least about 1 μg/ml, at least about 5 μg/ml, at least about 10 μg/ml, at least about 50 μg/ml, up to about 500 μg/ml, up to about 100 μg/ml.

Cavitation can be accomplished various methods, including without limitation by ultrasound treatment of the cells.

In some embodiments, ultrasound is applied for from about 10 to about 30 seconds, usually in bursts of 5 seconds; and may be applied for about 10, about 15, about 20, about 25, about 30 seconds. Following ultrasound, the cells may be vigorously pipetted from about 3 to about 20 times, e.g. about 3, about 5, about 8, about 10, about 12, about 15, about 20 times. The suspension of cells may be rested briefly between pipetting. Additional ultrasound may be performed following the pipetting. In one embodiment, ultrasound utilizes an energy output of from about 500 to about 2500 j of energy. Probe temperature may be set, for example at from about 50° to about 75° and may be about 65°. During this process, the solution may be observed to see that microbubbles are being generated. The media should become translucent with a noticeable decrease in viscosity. The microbubbles in the solution should dissipate in −30 seconds to 1 minute after sonication. A slight media color change should remain.

Following the mechanical pressures, the suspension of cells is heat shocked, to a temperature of at least about 45° C. and not more than about 80° C., and may be at about 45° C., at about 50° C., at about 60° C., at about 65° C., at about 70° C., at about 75° C., at about 80° C. The time of exposure may depend on the temperature, but is usually not more than about 5 minutes, not more than about 4 minutes, not more than about 3 minutes, not more than about 2 minutes, and may be from about 50 to about 75 seconds.

Immediately following heat shock, the cells are cold-shocked, conveniently by immersing in an ice bath. The temperature of the medium may be reduced to about 5° C.; to about 10° C., to about 15° C., to about 20° C., etc. The time of exposure may depend on the temperature, but is usually not more than about 5 minutes, not more than about 4 minutes, not more than about 3 minutes, not more than about 2 minutes, not more than about 1 minutes, and may be from about 1 to 5 minutes.

The treated cells are then transferred to growth medium, for example DMEM with high glucose. The medium may comprise one or more factors selected from: LIF, EGF, L-ascorbic acid, CHIR99021, TGF-β RI Kinase Inhibitor II, PD0325901, bFGF, IGF-I. The concentrations of EGF and L-ascorbic acid may be as described above. In some embodiments the medium comprises at least includes all of LIF, L-AA, CHIR99021, TGF-β RI Kinase Inhibitor II, PD0325901 and for human cells bFGF.

Concentration of LIF may be from 100-10,000 U/ml leukemia inhibitory factor, e.g. at least about 100 U/ml, at least about 500 U/ml, at least about 1000 U/ml, and up to about 5000 U/ml, up to about 10,000 U/ml.

Concentration of CHIR99021 may be at least about 0.5 μM, at least about 1 μM, at least about 3 μM, at least about 10 μM, up to about 50 μM, up to about 500 μM.

TGF-β RI Kinase Inhibitor II (Y-27632) may be present at a concentration of at least 10 nM, at least about 50 nM, at least about 100 nM, up to about 500 nM, up to about 750 nM, up to about 1 μM.

PD0325901 (MEK inhibitor) may be at least about 0.05 μM, at least about 0.5 μM, at least about 1 μM, at least about 10 μM, up to about 50 μM, up to about 500 μM.

IGF-I may be present at a concentration of from about 0.5 ng/ml to about 250 ng/ml, e.g. at least about 0.5 ng/ml, at least about 1 ng/ml, at least about 5 ng/ml, at least about 10 ng/ml, at least about 50 ng/ml, at least about 100 ng/ml or more.

bFGF for human cells may be present at a concentration of from about 0.5 ng/ml to about 250 ng/ml, e.g. at least about 0.5 ng/ml, at least about 1 ng/ml, at least about 5 ng/ml, at least about 10 ng/ml, at least about 50 ng/ml, at least about 100 ng/ml or more.

Cells may be maintained in this medium for at least 1 hour, at least 2 hours, at least 6 hours, at least 12 hours, at least 1 day, at least 2 days, at least 7 days or longer. By way of non-limiting example, other conditions suitable for propagation of pluripotent cells include plating cells at 10⁶ cells/ml in DMEM supplemented with 10-100 ng/mL epidermal growth factor, 10-100 ng/ml LIF, 5-10% FBS, etc. About 50% of the medium can be replaced every 2-3 days for the duration of the culture. During propagation, the pluripotent cell generated according to the methods described herein will continue to express the same pluripotent stem cell marker(s).

Cells thus cultured have certain markers of pluripotency. Non-limiting examples of pluripotent stem cell markers include SSEA-1, SSEA-2, SSEA-3, SSEA-4 (collectively referred to herein as SSEA), AP, E-cadherin antigen, Oct4, Nanog, Ecatl, Rexl, Zfp296, GDF3, Dppa3, Dppa4, Dppa5, Sox2, Esrrb, Dnmt3b, Dnmt31, Utfl, Tell, Batl, Fgf4, Neo, Cripto, Cdx2, and Slc2a3. Methods of determining if a cell is expressing a pluripotent stem cell marker are well known to one of ordinary skill in the art and include, for example, RT-PCR, the use of reporter gene constructs (e.g. expression of the Oct4-GFP construct described herein coupled with FACS or fluorescence microscopy), and FACS or fluorescence microscopy using antibodies specific for cell surface markers of interest.

Following the conditioning protocol, the resulting Epinul cells can be grown in culture for a variety of purposes and outcomes. The cells in culture can be directed to a differentiation pathway or pathways by the addition of factors and selection of media and growth conditions. In one embodiment, the Epinul cells differentiate to a blastocyst or morula state, from which pluripotent embryonic stem cells can be derived. In other embodiments the Epinul cells in culture are maintained in a pre-embryonic state.

In one embodiment, the one or more environmental pressures in the exposing step comprises cavitation. In another embodiment, the one or more environmental pressures in the exposing step comprises acoustic waves. In some embodiments, the one or more environmental pressures in the exposing step comprises extreme heat. In further embodiments, the one or more environmental pressures in the exposing step comprises passing the cells back and forth through a constrained region. In some embodiments, the one or more environmental pressures in the exposing step comprises atmospheric pressure changes. In other embodiments, the one or more environmental pressures in the exposing step comprises fluid viscosity changes. In some embodiments, the one or more environmental pressures in the exposing step comprises oxygen saturation. In other embodiments, the one or more environmental pressures in the exposing step comprises CO2 saturation. In some embodiments, the cells that have been exposed to one or more environmental pressures are further treated with proteinase K before the transferring step. In some embodiments, the proteinase K-treated cells are placed in a heat bath followed by an ice bath. In some embodiments, the high glucose media of the transferring step comprises LIF and/or vitamin C. In other embodiments, the high glucose media of the transferring step comprises one or more of buffering systems, inorganic salts, amino acids, carbohydrates, proteins and peptides, fatty acids and lipids, vitamins, trace elements, media supplements, antibiotics, and/or serum. In one embodiment, the high glucose media of the transferring step comprises glutamine. In another embodiment, the high glucose media of the transferring step comprises penicillin-streptomycin. In a further embodiment, the high glucose media of the transferring step comprises basic fibroblast growth factor (bFGF). In yet a further embodiment, the high glucose media of the transferring step comprises a chemically defined animal (xeno)-free and serum-free media.

In one embodiment, the sample is from a mammal. In another embodiment, the mammal is a human. In some embodiments, the sample is from one or more organs. In other embodiments, the sample is from one or more tissues.

In one embodiment, the Epinuls can become pluripotent stem cells. In another embodiment, the pluripotent stem cells are embryonic stem cells.

In another embodiment, cold media is added to produce a preferred range of working size and initial population of cells per sample. In this embodiment, a total of about 20 mL of cells, trypsin and media are separated into about 1 mL samples, approximately (number of starting cells) into 50 mL tubes for further treatment.

In another embodiment, a prepared aliquot of ATP suspended in DMEM is administered to the sample and vigorously titrated to catalyze early reactions and aid in enzymatic process. In some embodiments, other elements can be added that will assist, but are not required for success of the protocol. These elements merely support and assist in various ways. These elements can include, but are not limited to, L-ascorbic acid, retinoic acid, and the like.

In another embodiment, ITS (insulin transferrin selenium) and EGF (epidermal growth factor) diluted into DMEM are added to the sample and titrated through the sample to ensure maximal dispersion throughout the sample.

In another embodiment, one or more doses of fetal bovine serum (FBS) to saturate free amino acids into the sample environment can be added, e.g. at a concentration at from about 1% to about 20%, e.g. up to about 1%, up to about 2%, up to about 5%, up to about 10%, up to about 15%, up to about 20%.

In one embodiment, the sample is loaded into a device providing a series of predetermined acoustic waves of varying strengths in a cyclic manner to enact electromagnetic disruption of molecular bonds, sheer forces, velocity and extreme pressure changes to the environment. The cavitation or ultrasound process generates the formation of micro-bubbles and the successive destruction of these bubbles causes localized and extremely small regions of extreme heat, sometimes nearing 5,000 degrees kelvin. This disrupts higher order structures creating component parts, initiates the maximum heat shock response and initiates the BBNP survival cascade.

In another embodiment, optionally carried out utilizing vacuum and tapered cylinders, pulling the sample through a constrained region into a broader region, reversing the process and adding air pressure to force the sample back through the constrained region and repeating the process in a controlled and rapid flow to create the harmonics necessary to induce cavitation. The synthetic creation of the process shares similar characteristics in that manual velocity and sheer forces are created as well as severe pressure changes by forcing the sample through a constrained opening, and the expansion or flex is achieved by the sample passing into a larger area to disperse before pressure from the opposite direction compacts and forces the sample back through the constrained opening and flowing into the sample vessel.

In one embodiment, the color of the sample will change slightly as the process introduces micro-bubbles into the sample. An addition of a small amount of proteinase K to act as a chromatin digest is now added and cavitation is resumed for a short duration. In this embodiment, the samples are immediately removed and placed into a heat bath at 65° C. for 1-2 minutes depending upon the method used for cavitation. This neutralizes the proteinase and further denatures free cell products in the environment to be used in the reformation of lipid bi-layers, enzymes and cascade signaling molecules for available integration. This step again enforces the activation of HSP family and formation of additional chaperonins and co-activators and to fully strip lingering epigenetic modulations of the chromatin structure, leaving a naked or loose and pliable DNA structure.

In another embodiment, the sample is immediately removed from the heat and placed into an ice bath to rapidly cool the sample to prevent unwanted denaturing and to enact the opposite spectrum of the CSP/HSP support which will be the critical biological machinery that will rapidly produce temporary lipid rafts and loose membranes around fully primed DNA. At this point the required factors, machinery and chromatin state are fully prepped and the BBNP process will gather the elements from the surrounding environment to generate various vehicles for preservation and further expansion.

In a further embodiment, the samples are transferred to a growth vessel, and fed with prepared high glucose media that optionally include one or both of: LIF and ascorbic acid, and placed in incubation at 37° C. and 5% CO₂. In other embodiments, the high glucose media comprises one or more of buffering systems, inorganic salts, amino acids, carbohydrates, proteins and peptides, fatty acids and lipids, vitamins, trace elements, media supplements, antibiotics, and/or serum. In one embodiment, the high glucose media comprises glutamine. In another embodiment, the high glucose media comprises penicillin-streptomycin. In a further embodiment, the high glucose media comprises basic fibroblast growth factor (bFGF). In yet a further embodiment, the high glucose media comprises a chemically defined animal (xeno)-free and serum-free media.

### Systems for Preparing Conditioned Stem Cells

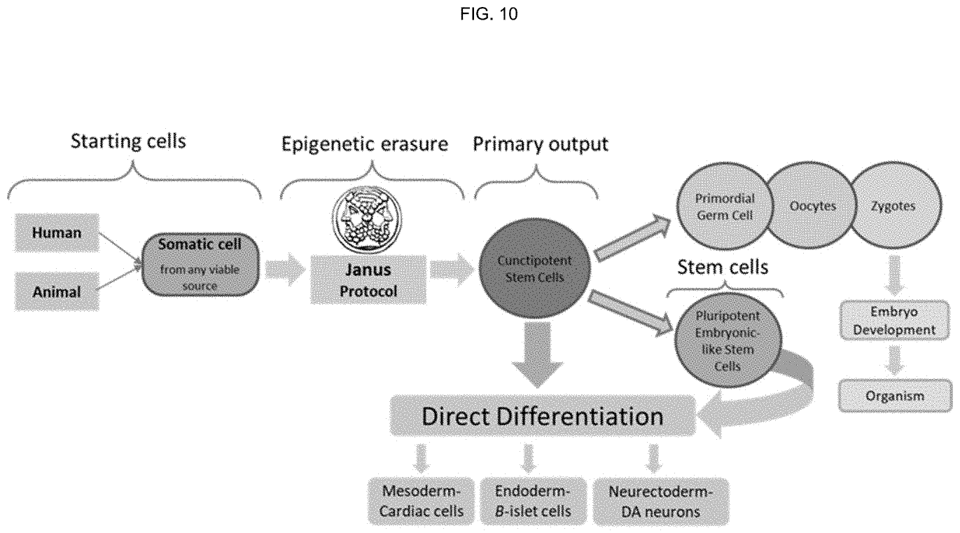

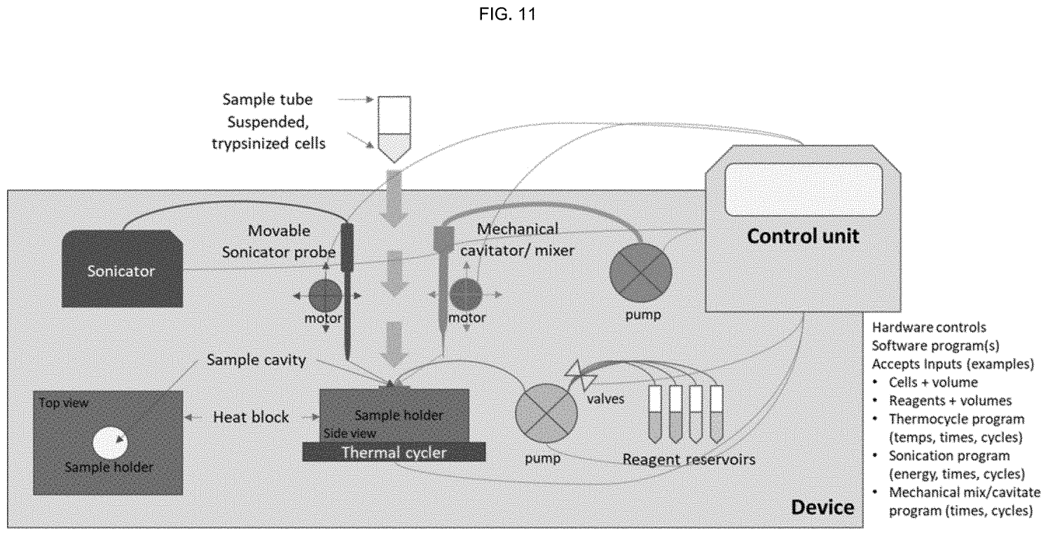

In one embodiment, a device is utilized for automation of the disclosed protocol, for example as depicted in FIG. 10. Automation of the mechanical and environmental influencers may be built into an automated and software configurable system. In some embodiments, the system is cGMP compliant, comprises a controlled environment, is sterile, and an exact replication. A cell sample, e.g. somatic mammalian cells, can be inserted into the device for epigenetic conditioning. Programmed product specifications can be set in software model.

Parameters of control may include: temperature, rate of change and time of each change, psi of environment and rate of change in either direction as well as electromagnetic, thermodynamics, amplitude, frequency, power and time at each interval. There may be automated flow of materials through constructed chambers—arrangement of various sized tubes with constrictions expanding into open wells followed by constriction into another open holding chamber. In some embodiments, the rate of flow through chambers, number of passages, time intervals between exchanges are program variables. This correlates directly to shear forces, heat, dispersion of environmental raw materials, cell signals, sonification and reactive oxygen species (ROS) generation. Cavitation chamber and controlled sound waves are controlled according to cell types and desired end product. Rate of compression and rebound, length of bombardment and force are all correlated and available as variable sets of parameters.

In other embodiments, the desired input of required chemicals and growth factors are added in specific doses. In one embodiment, media formats are programmed.

In some embodiments, variable parameters for growth to differentiated cells can be instituted—for example, cardiomyocytes may be grown in an oxygen depleted environment, with higher psi and specific electrical fluctuations to mimic being in a host, allowing for development of robust and primed cells.

In another embodiment, cells are seeded into growth containers of choosing within the enclosed and sterile environment and can either be transferred to a culture environment or shuttled to the internal controlled growth chambers.

In some embodiments, growth chambers are programmable environments—CO₂, O₂, psi, heat, electrical activity, administration of additional growth factors, feeding and automated passaging. In other embodiments, growth chambers vary according to desired product and include, but are not limited to, various growth containers, surface coatings for attached cells, bioreactors for suspension cells, and/or 3D chambers allowing for growth on defined matrix. n some embodiments, advanced system chambers include probes for measuring pH, glucose, etc.

In some embodiments, imaging of cells/tissues in chambers is done via built in visualization instruments.

Application of conditioned stem cells upon retrieval may include: induction of proliferation, cell expansion, differentiation into one or more specialized cell types, genetic modification, short- or long-term storage (e.g., cryopreservation or any method known in the art), screening, diagnostic probing, phenotyping, and therapeutic interventions such as for transplantation, chemotherapy, disease treatment, disease prevention, and cell, organ or tissue replacement or enhancement, as examples.

### Physical Automated System Design

Sonication system capable of delivering high intensity (20 kW/cm2) short pulses (20 microseconds) of energy to a target volume. A 650-kHz, phased-array ultrasound system that can deliver high-intensity (25 kW/cm2), ultrasound pulses up to (15 cycles in 20 ms) at pulse repetition frequencies of 100 Hz to 20 kHz Hz with acoustic pressures varying from 1 to 12 MPa peak negative pressure.

This will allow for precision removal or ablation of cellular targets, including directed and selective Sonication Parameters (For manual protocol—calculated to specific cell sources and defined number of cells in a 3 ml solution.)

Manual Protocol. The ultrasonic energy is produced from the tip and is directed downward. As the solution is being processed, the liquid is pushed in all directions. Based on the size and shape of the vessel, a 15 ml plastic conical tube used, a ¼″ microtip is used for 3 ml volume. (Calculations take into account the variance in work and magnetic properties from the center of the tube directly in contact to the edges. This in fact creates a part of the variable environment of disruption to cells, destruction of cells and the denaturing of proteins to “seed” the solution with desired factors and raw materials.)

Specifications: Current protocol. Power: 700 watts—measure of energy per unit of time that is conveyed from the generator to the sonicated liquid measured in watts (W) or kilowatts (KW) delivered to the sonicated liquid via a unit (cm\hat{ }2) of the horn's radiating area. Frequency: 19-21 kHz will give 19-21,000 vibration cycles per second. Amplitude: 37 dB

The effectiveness of ultrasonic processing is directly related to the intensity, (amplitude and intensity have a direct relationship) homogeneity and size of the cavitation field created in the liquid, thus you must calculate the “solution” properties as an additional part of the calculations for the engineered epigenetic disruptions desired.

The 15 ml conical tubes are filled with a solution of cells, media and additives to a level of 3 ml—3 ml being the current maximum size. Sonication is performed in a series of 3 steps, beginning with sonication and cavitation via a probe inserted into the mixture, going from the top, nearly touching the base and returning up and out of the solution. Total time for this calculated protocol is three 5 second bursts. Amplitudes are variable for different cell sources and densities—for this application, amplitude is set to 37 dB, frequencies between 16 kHz and 20 kHz, with power 45 W producing 1,350 j of energy. Probe temperature is set to 65° C. for 17 seconds with pulses from 1-5, 7-11, 12-17 seconds (2 one second interrupts)

This work also involves calculations for dynamic and energy-flow modeling, aspects of nonlinear dynamics of ultrasonic cavitation applied to disruption of molecular bonds and frequency ablation/diffusion. The sonication interaction with the fluid involves compression and rarefaction phenomena leading to cavitation of the fluid. It is important not to overlook the energetics involved in the overall process. This involves the dynamic and energy-flow modeling aspects of nonlinear dynamics of ultrasonic cavitation applied to disruption of molecular bonds and frequency ablation.

Acoustics theory. When liquids are exposed to ultrasonic waves, acoustic cavitation occurs, which includes rapid formation, growth, and collapse of bubbles. Local energies associated with acoustic cavitation are equivalent to 5000° C. and 1,000 atm pressures.

### Expansion of Cells in Culture

For inducing proliferation, the culture medium may be generally supplemented with at least one proliferation-inducing substituent or substance (i.e., a chemical or biologic factor, generally trophic, that includes cell division, such as molecules that binds to a receptor on the surface of the cell and exert trophic or growth-inducing effects). Examples of proliferation-inducing growth factors include EGF, amphiregulin, FGFs, TGFs, as examples, used alone or in combination. Additional substituents may be added to the culture medium, especially those that are lineage specific substituents such as vitamins, NGF, PDGF, TRH, TGF, BMP, GM-CSF, or IGF, as examples.

Proliferating cells will continue to proliferate in suspension if continually offered the appropriate culture medium as described above. The proliferating cells in culture may also be passaged and proliferation reinitiated. Importantly, passaging and reinitiating proliferation may be continuously repeated (e.g., weekly) resulting in a logarithmic increase in the number of cells after each passage.

### Differentiation of Cells

Differentiation of cells of the present disclosure may be induced by any method that activates the cascade of biological events leading to growth, including such things as liberating inositol triphosphate (ITP), intracellular Ca2+, liberation of diacyl glycerol and/or the activation of protein kinase C (PKC) and other cellular kinases, as examples. Differentiation is controlled by external signals, such as chemical secretions by other cells, physical contact with neighboring cells, and certain other molecules in the environment (e.g., molecular substituents). Other examples of methods that induce differentiation include treatment with phorbol esters, differentiation-inducing growth factors and other chemical signals, alone, in a temporal sequence or in combination with other signals. In addition, plating the cells on a fixed substrate (e.g., flask, culture plate, or coverslip that may also be coated with an ionically charged surface such as poly-L-lysine and poly-L-omithine, as examples) may also induce differentiation. Other substrates that may induce differentiation include those that resemble the extracellular matrix such as collagen, fibronectin, laminin, as examples and may be used alone or in combination. Differentiation may also be induced in a suspension in the presence of a proliferation-inducing substituent.

After addition of the appropriate differentiation-inducing agent(s) to cells prepared by methods of the present disclosure, many of the cells will differentiate. Differentiation and detection of a specific cell-type or lineage may be determined by morphology, immunocytochemistry and/or immunohistochemical methods or by expression of cell-type specific RNA or DNA. For example, cell-type specific antibodies, expression of specific genes, or specific histochemical assays may be used to distinguish cellular characteristics or phenotypic properties of the specialized cells. Those skilled in the art will be able to recognize the methodologies that best characterize a specific cell type. Cells may specialize into one of any cell type depending on the substituent (or temporal sequence thereof) that is added to the cell culture medium. Examples of cell types include neural (e.g., glia, dendrites, etc.), non-neural (astrocytes, oligodendrocytes), epithelial, hematopoietic, hepatic, cardiac, endothelial, muscular (smooth and skeletal), epidermal, osteoblastic, osteoclastic, chondrocytic, stromal, adipocytic, as examples.

### Genetic Modification and Manipulation

The Epinul cells possess features of a continuous cell line. In the unspecialized state, in the presence of a proliferation-inducing substituent, cells continuously divide and are, therefore, excellent targets for genetic modification. The term “genetic modification” or “genetic manipulation,” as used herein, refers to the stable or transient alteration of the genotype of conditioned stem cells by intentional introduction of exogenous nucleic acid. The nucleic acid may be synthetic or naturally derived, and may contain genes, portions of genes, or other useful nucleic acid sequences.

Exogenous nucleic acid may be introduced to a stem cell of the present disclosure by various methods known in the art, and including without limitations zinc finger domain, CRISPR-Cas9 and other methods of introducing very specific recombination repair of DNA lesions viral vectors (retrovirus, modified herpes viral, herpes-viral, adenovirus, adeno-associated virus, cytomegalovirus as examples) or mammalian cell-specific promoter or transfection (lipofection, calcium phosphate transfection, DEAE-dextran, electroporation, as examples) that direct the expression of one or more genes encoding a desired protein and may be used to promote differentiation. As used herein, the protein may be any protein or protein combination of interest and may be linked to a selectable marker for detection. In addition, the vectors may include a drug selection marker.

An alternative approach is the intentional immortalization of the stem cell by introducing an oncogene that alters the genetic make-up of the cell thereby inducing the cell to proliferate indefinitely. In addition, stem cells, especially those induced to differentiate can be genetically modified to cease cell death by administering Bcl-2 or by genetically modifying the cells with the bcl-2 gene, whose product is known to prevent programmed cell death (apoptosis).

The disclosure further involves a therapeutically effective dose or amount of Epinuls applied to damaged tissue of an organ. An effective dose is an amount sufficient to effect a beneficial or desired clinical result. Said dose could be administered in one or more administrations. An effective dose of somatic stem cells may be from about 2×10⁴ to about 2×10⁷, about 1×10⁵ to about 6×10⁶, or about 2×10⁶. The precise determination of what would be considered an effective dose may be based on factors individual to each patient, including their size, age, type of organ to be treated, area and severity of the damaged tissue, and amount of time since damage. One skilled in the art, specifically a physician, would be able to determine the number of Epinuls that would constitute an effective dose without undue experimentation. In another aspect of the disclosure, the Epinuls are delivered to the organ.

Further embodiments of the disclosure require the conditioned, small totipotent pre-embryonic stem cells to migrate into the damaged organ tissue and differentiate into cell lineages that comprise that organ. Differentiation into one or more cell lineages of the organ to be repaired is important for at least partially restoring both structural and functional integrity into the damaged tissue.

### Therapeutic Uses and Other Applications of Conditioned Stem Cells

Today, donated organs and tissues are often used to replace ailing or destroyed tissue, but the need for transplantable tissues and organs far outweighs the available supply. Stem cells, directed to differentiate into specific cell types, offer the possibility of a renewable source of replacement cells and tissues to treat diseases including macular degeneration, spinal cord injury, stroke, burns, heart disease, diabetes, osteoarthritis, and rheumatoid arthritis through cell transplantation and replacement therapies. Stem cell therapy can be viewed as a promising option in two different ways. The first is as a “support” mechanism, in which stem cells are exploited to promote complete tissue repair and avoid detrimental fibrosis. The other is the “replace” option, in which stem cells differentiate and substitute for damaged cells, providing an alternative to organ transplantation.

Stem cell-based therapies could be used to cure multiple inherited and degenerative disorders as well as adjuvant immunotherapy for treating patients diagnosed with immune system deficiencies and refractory/relapsed cancers for which there are few or no cures. Degenerative disorders and diseases include, but are not limited to, hematopoietic and immune system disorders, cardiovascular diseases, diabetes, chronic hepatic injuries, gastrointestinal disorders, brain, eye, and muscular degenerative diseases, and aggressive cancers.

In one embodiment, a Epinul cell is generated that is autologous or HLA-matched to a recipient. In some embodiments the Epinul cell is differentiated along a pre-defined cell lineage prior to administering the cell or tissue to the recipient; in other embodiments a pluripotent cell, e.g. a Epinul cell or pluripotent progeny is administered, permitting in vivo differentiation to the desired cell type with or without the administration of agents to promote the desired differentiation. Treatment with autologous cells may include the step of correcting undesirable genetic traits, e.g. mutations that cause disease.

Transplantation may be accompanied by immunosuppression as deemed necessary by one of ordinary skill in the art. In some cases, stem cells with genetic modification that includes gene replacement or gene knockout using homologous recombination may be employed. For example, techniques for ablation of major histocompatibility complex (MHC) genes are well known in the art (Zheng et al., 1991. PNAS, 88:8067-8071). Stem cells lacking MHC expression allows the use of cells of the present disclosure across allogeneic and xenogeneic histocompatibility barriers without the need to immunosuppress.

Diseases that may be treated include, without limitation, aplastic anemia, Fanconi anemia, and paroxysmal nocturnal hemoglobinuria (PNH). Lysosomal storage diseases, including mucopolysaccharidoses (MPS), Hurler's syndrome (MPS-I H), Scheie syndrome (MPS-IS), Hunter's syndrome (MPS-II), Sanfilippo syndrome (MPS-III), Morquio syndrome (MPS-IV), Maroteaux-Lamy Syndrome (MPS-VI), Sly syndrome, beta-glucuronidase deficiency (MPS-VII), adrenoleukodystrophy, mucolipidosis II (I-cell Disease), Krabbe disease, Gaucher's disease, Niemann-Pick disease, Wolman disease and metachromatic leukodystrophy. Also treatable with stem cell therapy are: lung disorders, including COPD and bronchial asthma; congenital immune disorders, including ataxia-telangiectasia, Kostmann syndrome, leukocyte adhesion deficiency, DiGeorge syndrome, bare lymphocyte syndrome, Omenn's syndrome, severe combined immunodeficiency (SLID), SLID with adenosine deaminase deficiency, absence of T & B cells SLID, absence of T cells, normal B cell SLID, common variable immunodeficiency and X-linked lymphoproliferative disorder; other inherited disorders, including Lesch-Nyhan syndrome, cartilage-hair hypoplasia, Glanzmann thrombasthenia, and osteopetrosis; neurological conditions, including acute and chronic stroke, traumatic brain injury, cerebral palsy, multiple sclerosis, amyotrophic lateral sclerosis and epilepsy; cardiac conditions, including atherosclerosis, congestive heart failure and myocardial infarction; metabolic disorders, including diabetes; and ocular disorders including macular degeneration and optic atrophy. Cancers may be treated by high dose ablative therapy followed by hematopoietic stem cell transplantation.

In one embodiment, the Epinul cells are differentiated into neural progenitors. In another embodiment, these neural progenitors are injected into the region of the frontal cortex that contains these stem cells and is responsible for new neuron formation. As these stem cells are greatly depleted as an individual ages, the stem cells of the present disclosure can provide a path to plasticity and expanded learning.

In another embodiment, the conditioned stem cells of the present disclosure are used to regenerate whole organs, organ systems or limbs. In some embodiments, the organs or organ systems include, but are not limited to, thymus, adrenal gland, thyroid gland, intestine, lungs, heart, liver, blood vessels, germ cells, nervous system, eye tissues, hair cells, kidney and bladder, skin, hair follicles, pancreas, bone, and cartilage. In yet another embodiment, the conditioned stem cells of the present disclosure are used to repair spinal discs and bone fractures. In a further embodiment, the conditioned stem cells of the present disclosure are used to replace damaged myelin sheath.

In one embodiment of the present disclosure, transplantation with Epinul cells or progeny derived therefrom is performed in order to treat or prevent one or more disorders, diseases, or degenerative conditions, to repair or replace a damaged, poorly functioning or malfunctioning tissue/organ, or to enhance cell, tissue, or organ function. Such organ or tissue injuries (also referred to as affected areas) may be from mechanical, chemical, molecular, or electrolytic insults, changes or abnormalities.

Cells can be delivered throughout the affected area or to one or more specific sites as deemed appropriate to one of ordinary skill in the art. The cells are administered using any method known to maintain the integrity of organ or tissue. In one embodiment of the present disclosure, transplanted stem cells or specific components of the cells have been genetically modified to include one or more tracers (e.g., dyes or markers such as rhodamine- or fluorescein-labeled microspheres, or fast blue, bisbenzanide, or retrovirally introduced histochemical markers, or isotopic compounds, or light-modifiable chemicals or proteins such as green fluorescence protein, as examples). Here, Epinul cells can be used as diagnostic markers, for probing, for visualization of tissue or organ remodeling changes, for markers of response to one or more stimuli (e.g., mechanical or chemical, such as electric fields, proteins, chemicals, drugs, etc.) or other therapeutic purposes.

Epinul cells offer the unique opportunity to assess the quality of disease-relevant cell types by directly comparing cells derived in vitro by the protocol of the present disclosure with their genetically identical in vivo counterparts. For instance, if the aim is to transplant conditioned small pre-embryonic totipotent stem cell-derived blood cells back into an individual, there will be an ability to determine how similar the cells derived in vitro are to blood cells isolated from that same individual.

In one aspect of the disclosure, a method is provided for treating or preventing a disease or disorder in a subject in need thereof, comprising: (a) obtaining somatic cells from the subject in need thereof; (b) conditioning the somatic cells in vitro to become Epinul cells; (c) expanding the Epinul cells; and (d) administering said expanded Epinul cells to the subject in need thereof, wherein the Epinul cells generate differentiated cells that assemble into one or more new tissues or organs following the administration, thereby treating or preventing a disease or disorder in a subject in need thereof.

In another aspect of the disclosure, a method is provided for repairing and/or regenerating one or more damaged tissues or organs in a subject in need thereof comprising: (a) obtaining non-damaged somatic cells from the subject in need thereof; (b) conditioning the somatic cells in vitro to become Epinul cells; (c) expanding the Epinul cells; and (d) administering said expanded Epinul cells to the subject in need thereof, wherein said Epinul cells generate differentiated cells that assemble into one or more new tissues or organs following the administration, thereby repairing and/or regenerating the one or more damaged tissues or organs.

### Additional Methods of Use

A pharmaceutical composition for use in treating or preventing a disease or disorder in a subject in need thereof is provided comprising a population of Epinul cells. In some embodiments the Epinul cells are autologous to an intended recipient. In some embodiments the Epinul cells are MEW matched to an intended recipient. The cells may be provided in a therapeutically effective dose.

An effective dose is an amount sufficient to effect a beneficial or desired clinical result. Said dose could be administered in one or more administrations. An effective dose of Epinuls may be from about 10⁴ to about 10⁹, about 1×10⁵ to about 10⁸, from about 10⁶ to about 10⁸; from about 10⁷ to about 10⁹, etc. The precise determination of what would be considered an effective dose may be based on factors individual to each patient, including their size, age, type of organ to be treated, area and severity of the damaged tissue, and amount of time since damage. One skilled in the art, specifically a physician, would be able to determine the number of Epinuls that would constitute an effective dose without undue experimentation.

The Epinuls may be delivered to a target organ. In some embodiments, the Epinuls are delivered specifically to the border area of an infarcted region of the organ. As one skilled in the art would be aware, the infarcted area is visible grossly, allowing this specific placement of stem cells to be possible.

Further embodiments of the disclosure require the Epinuls to migrate into the damaged organ tissue and differentiate into cell lineages that comprise that organ. Differentiation into one or more cell lineages of the organ to be repaired is important for at least partially restoring both structural and functional integrity into the damaged tissue. Another embodiment of the disclosure includes the proliferation of the differentiated cells and the formation of the cells into organ structures.

In one embodiment, Epinul cells are used for bioengineering purposes. In one embodiment, the stem cells of the present disclosure are recruited in a three-dimensional matrix (e.g., scaffold) that represents the tissue of interest. Upon recruitment, stem cells may or may not be induced to differentiate. The stem cells of the present disclosure may remain in the scaffold, where they are genetically modified or induced to differentiate or may be removed from the scaffold where they undergo further manipulation prior to use. Importantly, the stem cells of the present disclosure may be recruited and induced to differentiate in vivo at the site of interest in order to create a new organ and/or tissue. Alternatively, the scaffold is retrieved and implanted elsewhere or donated to a recipient.

In other embodiments, methods are provided for the treatment of cancer and other diseases where the proximal cause of the disease is the accumulation of one or more epigenetic signals altering gene transcription resulting in aberrant cells or cell function. In such methods, the production of Epinul cells erases the accumulated epigenetic signals, restoring healthy cell function, allowing the cells to be transplanted back into the patient restoring health. In some such embodiments, the cancer is a hematologic cancer, e.g. leukemia, lymphoma, myeloma, etc., where cancer cells are obtained from the patent, treating with the Janus protocol to erase the epigenetic signals, expanded, differentiated into hematologic progenitor cells, then reintroduced to the patient after their own hematologic stem cells are ablated by chemical agents or radiation.

Another regenerative medicine embodiment comprises the steps of taking a sample of cells from a patient, creating Epinul cells, expanding these cells, then reintroducing them into the patient. This acts as the equivalent of parabiosis, where the blood or plasma of young animals reinvigorates old animals.

In another embodiment, Epinul cells are used for disease modeling. While animal models have been crucial in the investigation of disease mechanisms, fundamental developmental, biochemical, and physiological differences exist between any non-human animal model, including other primates, and humans. The importance of utilizing human cells for these purposes is evidenced by the large numbers of failed clinical trials, which are at least partly attributed to these species differences. The concept of utilizing ES and iPS cells to model a disease in a culture dish is based on the unique capacity of these cells to continuously self-renew and their potential to give rise to all cell types in the human body. Thus, Epinul cells provide a limitless reservoir of cell types that in many cases were not previously possible to obtain, for example, the motor and dopaminergic neurons affected in amyotrophic lateral sclerosis (ALS) and Parkinsons' Disease (PD). The advantage of the protocol of the present disclosure is that it allows for the generation of totipotent cells from any individual in the context of his or her own particular genetic identity, including individuals with sporadic forms of disease and those affected by complex multifactorial diseases of unknown genetic identity, such as autism spectrum disorders and type 1 diabetes.

In another embodiment, Epinul cells, either those induced to differentiate or not, will be essential for screening of toxins and of potential therapeutic compositions and pharmaceutical preparations. The unique properties of conditioned, small pre-embryonic totipotent stem cells of the present disclosure also provide for practical approaches in pharmaceutical toxicology and pharmacogenomics. In particular, hepatotoxicity and cardiotoxicity are two principal causes of drug failure during preclinical testing, while the variability in individual responses to potential therapeutic agents is also a major problem in effective drug development. The advantage of the protocol of the present disclosure is that it allows for the generation of a library of cell lines that may to a substantial extent represent the genetic and potentially epigenetic variation of a broad spectrum of the population. The use of this tool in high-throughput screening assays could allow better prediction of the toxicology caused by and therapeutic responses induced by newly developed dugs and offer insight into the underlying mechanisms. The net result of this approach would substantially decrease the risk and cost associated with early-stage clinical trials and could lead toward a more personalized approach in drug administration.

In one embodiment, compositions are applied to the cells of the present disclosure in vitro at varying dosages and times during proliferation and/or differentiation and cell response is likewise monitored over time as is well known in the art. Morphologic (physical), genetic, secretory, conductivity (e.g., ion channels or nerve conduction) and other such responses are analyzed by one or more methods well known in the art (e.g., Western blot, Southern blot, Northern blot gene screening, immunohistochemistry, protein, receptor and enzyme assays, enzyme-linked immunosorbant assays (ELISA), electrophoresis analysis, HPLC, radioimmune assays, electrophysiologic measures, as general examples). Similarly, cell type-specific or proliferating stem cells of the present disclosure may be grown on a feeder layer (acting as a substrate) or in a three-dimensional network. Thus, stem cells, prior to screening, may have already undergone differentiation.

Similar to in vitro screenings and testings, transplanted stem cells of the present disclosure (with or without the induction to differentiate) in the absence and presence of one or more specific compositions or preparations are observed for their efficacy and safety (e.g., host survival, pharmacologic, biochemical and immunologic effects, etc.). In addition, the stem cells or type-specific cells of the present disclosure are used to measure the effect of an implant or another transplant on cells or a host.

The term “potential therapeutic compositions” or “pharmaceutical preparations” refer to any agent, such as a chemical, polymer, radioactive substance, virus, protein, peptide, amino acid, lipid, carbohydrate, nucleic acid, nucleotide, drug, pro-drug, implant, and device, as examples. With the present disclosure, cells that have already been induced to specialize prior to the screening are also screened.

Screening with Epinul cells (either in vitro or in vivo and with or without further induction to differentiate) provides an economic way to test and monitor industrial or biologic chemicals and compounds. In one embodiment, cells are used for rapid identification of substances (e.g., via high throughput screening methods) involved in the proliferation, differentiation and survival of in vitro or host cells (including an organ or tissue). Furthermore, cDNA libraries may be constructed from stem cell or lineage-specific cells of the present disclosure using techniques known in the art. As such, nucleic acids or their factors involved in cell regulation, dysfunction, repair, remodeling, etc. are analyzed and industrial compositions and/or pharmaceutical preparations are designed to promote positive cell features and counteract negative ones. Diagnostic probes are also developed, especially those that identify one or more genetic disorders or dysfunction. In addition, cells of the present disclosure are investigated for their ability to secrete or produce potential therapeutic or industrial compositions.

In another embodiment, Epinul cells can be additionally used in the following non-limiting areas: therapeutic cloning, gene editing/gene therapy utilizing pre-embryonic cell lines, hair regeneration, animal cloning, food production from animal cells, and creation of plasma and blood banks.

### Cell Compositions

In another aspect of the disclosure, a pharmaceutical composition is provided comprising a population of Epinul cells; and a pharmaceutical acceptable carrier. The cell composition may be substantially homogenous. As described herein, the Epinuls are capable of generating one or more or all of the cell lineages of any organ or even are capable of generating a whole organism. The organ from which Epinuls can be prepared include, but are not limited to, heart, kidney, liver, spleen, pancreas, intestine, lung, stomach, brain, retina, esophagus, bladder, epidermis, or bone marrow.

The pharmaceutical compositions of the present disclosure may be used as therapeutic agents—i.e. in therapy applications. As herein, the terms “treatment” and “therapy” include curative effects, alleviation effects, and prophylactic effects.

In one embodiment, the pharmaceutical composition of the present disclosure is delivered via injection. These routes for administration (delivery) include, but are not limited to subcutaneous or parenteral including intravenous, intraarterial, intramuscular, intraperitoneal, intramyocardial, transendocardial, trans-epicardial, intranasal administration as well as intrathecal, and infusion techniques.

The pharmaceutical composition can include suitable excipients, or stabilizers, and can be, for example, solutions, suspensions, gels, or emulsions. Typically, the composition will contain from about 0.01 to 99 percent, preferably from about 5 to 95 percent of cells, together with the carrier. The cells, when combined with pharmaceutically or physiologically acceptable carriers, excipients, or stabilizer, can be administered parenterally, subcutaneously, by implantation or by injection. For most therapeutic purposes, the cells can be administered via injection as a solution or suspension in liquid form. The term “pharmaceutically acceptable carrier” refers to a carrier for administration of the pluripotent cell generated according to the methods described herein and/or the at least partially differentiated progeny of the pluripotent cell. Such carriers include, but are not limited to, saline, buffered saline, dextrose, water, glycerol, and combinations thereof. Each carrier must be “acceptable” in the sense of being compatible with the other ingredients of the formulation, for example the carrier does not decrease the impact of the agent on the subject. In other words, a carrier is pharmaceutically inert and compatible with live cells.

Suitable formulations also include aqueous and non-aqueous sterile injection solutions which can contain anti-oxidants, buffers, bacteriostats, bactericidal antibiotics and solutes which render the formulation isotonic with the bodily fluids of the intended recipient. Aqueous and non-aqueous sterile suspensions can include suspending agents and thickening agents. The formulations can be presented in unit-dose or multi-dose containers.

Examples of parenteral dosage forms include, but are not limited to, solutions ready for injection, suspensions ready for injection, and emulsions. Parenteral dosage forms can be prepared, e.g., using bioresorbable scaffold materials to hold pluripotent cells generated according to the methods described herein and/or the at least partially differentiated progeny of the pluripotent cell.

When administering a pharmaceutical composition of the present disclosure parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for the compositions.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present disclosure, however, any vehicle, diluent, or additive used would have to be compatible with the autologous, genetically identical, pre-embryonic totipotent stem cells (Epinuls).

Sterile injectable solutions can be prepared by incorporating the compounds utilized in practicing the present disclosure in the required amount of the appropriate solvent with various amounts of the other ingredients, as desired.

The pharmaceutical composition of the present disclosure, e.g., comprising a therapeutically effective amount of Epinuls, can be administered to the patient in an injectable formulation containing any compatible carrier, such as various vehicles, adjuvants, additives, and diluents; or the compositions utilized in the present disclosure can be administered parenterally to the patient in the form of slow-release subcutaneous implants or targeted delivery systems such as monoclonal antibodies, iontophoretic, polymer matrices, liposomes, and microspheres. Known techniques which deliver the compositions orally or intravenously and retain the biological activity are preferred.

It is noted that humans are treated generally longer than the mice or other experimental animals which treatment has a length proportional to the length of the disease process and drug effectiveness. The doses may be single doses or multiple doses over a period of several days. Thus, one can scale up from animal experiments, e.g., rats, mice, and the like, to humans, by techniques from this disclosure and documents cited herein and the knowledge in the art, without undue experimentation.

The treatment generally has a length proportional to the length of the disease process and drug effectiveness and the patient being treated.

The quantity of the pharmaceutical composition to be administered will vary for the patient being treated and the type of organ to be treated. However, the precise determination of what would be considered an effective dose may be based on factors individual to each patient, including their size, age, organ to be treated, area and severity of the damaged tissue, and amount of time since damage. Therefore, dosages can be readily ascertained by those skilled in the art from this disclosure and the knowledge in the art. Thus, the skilled artisan can readily determine the amount of compositions and optional additives, vehicles, and/or carrier in compositions and to be administered in methods of the disclosure. Typically, any additives (in addition to the Epinuls) are present in an amount of 0.001 to 50 wt % solution in phosphate buffered saline, and the active ingredient is present on the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Examples of compositions comprising a therapeutic of the disclosure include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the disclosure, are conveniently provided as liquid preparations, e.g., isotonic aqueous solutions, suspensions, emulsions or viscous compositions which may be buffered to a selected pH. If digestive tract absorption is preferred, compositions of the disclosure can be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers can preferably dispense a metered dose or, a dose having a particular particle size.

Compositions of the disclosure can contain pharmaceutically acceptable flavors and/or colors for rendering them more appealing, especially if they are administered orally. The viscous compositions may be in the form of gels, lotions, ointments, creams and the like (e.g., for transdermal administration) and will typically contain a sufficient amount of a thickening agent so that the viscosity is from about 2500 to 6500 cps, although more viscous compositions, even up to 10,000 cps may be employed. Viscous compositions have a viscosity preferably of 2500 to 5000 cps, since above that range they become more difficult to administer. However, above that range, the compositions can approach solid or gelatin forms which are then easily administered as a swallowed pill for oral ingestion.

Liquid preparations are normally easier to prepare than gels, other viscous compositions, and solid compositions. Additionally, liquid compositions are somewhat more convenient to administer, especially by injection or orally. Viscous compositions, on the other hand, can be formulated within the appropriate viscosity range to provide longer contact periods with mucosa, such as the lining of the stomach or nasal mucosa.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form), or solid dosage form (e.g., whether the composition is to be formulated into a pill, tablet, capsule, caplet, time release form or liquid-filled form).

Solutions, suspensions and gels normally contain a major amount of water (preferably purified water) in addition to the active compound. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions can be isotonic, i.e., they can have the same osmotic pressure as blood and lacrimal fluid.

The desired isotonicity of the compositions of this disclosure may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

A pharmaceutically acceptable preservative can be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems can be readily avoided by reference to standard texts or by simple experiments (not involving undue experimentation), from this disclosure and the documents cited herein.

The inventive compositions of this disclosure are prepared by mixing the ingredients following generally accepted procedures. For example, the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally, the pH may be from about 3 to 7.5. Compositions can be administered in dosages and by techniques well known to those skilled in the medical and veterinary arts taking into consideration such factors as the age, sex, weight, and condition of the particular patient, and the composition form used for administration (e.g., solid vs. liquid). Dosages for humans or other mammals can be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

The pharmaceutical compositions of the present disclosure are used to repair and/or regenerate damaged organ tissue resulting from acute tissue injury (e.g., ischemic, toxic, immune-related insults) or various chronic degenerative disease. Accordingly, the disclosure involves the administration of Epinuls as herein discussed for the treatment or prevention of any one or more of these conditions as well as compositions for such treatment or prevention, use of Epinuls as herein discussed for formulating such compositions, and kits involving Epinuls as herein discussed, for preparing such compositions and/or for such treatment, or prevention. And advantageous routes of administration involve those best suited for treating these conditions, such as via injection, including, but are not limited to subcutaneous or parenteral including intravenous, intraarterial, intramuscular, intraperitoneal, intramyocardial, transendocardial, trans-epicardial, intranasal administration as well as intrathecal, and infusion techniques.

### Epigenetic Regulation of Stem Cells

DNA methylation maintains long-lasting cell memories, and is therefore considered to be a pivotal epigenetic barrier to cellular reprogramming. During reprogramming, the activation of endogenous pluripotency genes including Oct3/4 and Nanog is accompanied by erasing the methylation of cytosines at their promoter regions. Insufficient DNA demethylation at the promoter regions, which is occasionally observed in partially reprogrammed iPS cells, fails to produce the robust reactivation of pluripotency genes. In addition, the differential patterns of DNA methylation that are associated with genomic imprinting, retrotransposon silencing and X chromosome inactivation are observed between differentiated and pluripotent stem cells and among a series of pluripotent stem cell lines, indicating that DNA methylation may be a suitable epigenetic marker for characterizing pluripotent stem cell lines. Although it is unclear how such differential levels of DNA methylation arise, functional linkage between DNA methylation and reprogramming has been demonstrated. The inhibition of DNA methylation by chemical compounds or RNA interference targeting DNA methyltransferase can increase the efficiency of iPS cell generation.

Recent analyses using a high-performance sequencer have enabled mapping of DNA methylation with high resolution and have revealed an intriguing distribution of methylated cytosine in pluripotent stem cells. Since DNA methylation is frequently observed at CpG islands, which contain a high frequency of CpG sites, it is considered that the frequency of CpG sequences was positively correlated with the susceptibility to DNA methylation. However, the most recent studies of genome-wide DNA methylation status in pluripotent stem cells have produced observations that differ from the widely accepted model. The methylation levels of CpGs in pluripotent stem cells were negatively correlated with the local CpG density. In, where it was found that in ES and iPS cells, regions with high CpG density exhibited low DNA methylation, whereas those with low CpG density exhibited high DNA methylation. In contrast, regions with low CpG density are frequently observed in the promoters of tissue-specific genes, implying that the mechanism responsible for DNA methylation in the regulation of tissue-specific genes is different from that for DNA methylation in the regulation of other genes. Intriguingly, DNA hypermethylation at the promoters of these tissue-specific genes with low CpG density is accompanied by bivalent chromatins in ES and iPS cells. The relevance of this uniquely low CpG methylation level in pluripotent stem cells with bivalent domains is yet to be investigated at the molecular level. Surprisingly, approximately one-quarter of all methylated cytosines in ES and iPS cells occurred in a non-CpG context, whereas most of the methylated cytosines in somatic cells were observed in CpG sequences.

The capacity of embryonic stem (ES) cells to respond to differentiation stimuli and acquire a particular cell fate might may be determined by a very specific epigenetic trait known as bivalent chromatin. Bivalent chromatin domains that are enriched in histone H3 tri-methylated and di/tri-methylated at lysines 4 and 27 (H3K27me3 and H3K4me2/me3), respectively. H3K27me3 and H3K4me are marks associated with transcriptionally inactive and active chromatin, respectively. These opposing marks are thought to provide bivalent genes, which that are expressed at basal levels in stem cells, with the plasticity to reach full expression potential or be repressed upon activation of specific differentiation programs. Indeed, many of the genes in bivalent domains encode for transcription factors directing tissue-specific differentiation programs. This chromatin organization suggests that histone modifiers inducing H3K27me3 and H3K4me3 have a key function in maintaining pluripotency.

The Polycomb group (PcG) complexes with the activity of H3K27 methylation to repress the expression of developmentally regulated genes in pluripotent stem cells, whereas the Trithorax group (TrxG) complexes with the activity of H3K4 methylation to activate the expression of genes associated with self-renewal. H3K4me3 is frequently observed in promoter regions of pluripotent stem cells, and is linked to transcriptional activation in general. The methylation of H3K4 is mediated by TrxG members such as Set/mixed lineage leukemia (MLL) methyltransferases. H3K4 demethylase LSD1 stabilizes global DNA methylation and also maintains an appropriate balance between H3K4 and H3K27 methylation in the regulatory regions of several developmental genes in pluripotent stem cells.

Importantly, bivalent chromatin is not the only epigenomic trait associated with ES cells. Epigenetic silencing associated with histone lysine 9 methylation also contributes to stem cell maintenance. It is known that globally, H3K9me2 and H3K9me3 histone marks, associated with repressive chromatin, are maintained at low levels in ES and they become enriched in differentiated cells. The H3K9me demethylases Jmjd1a and Jmjd2c are important for ES cell self-renewal. Notoriously, Oct4 positively regulates the expression of these histone demethylases, which maintain the Tcl1 and Nanog genes (two key transcription factors for self-renewal in ES cells) in an open chromatin configuration by H3K9me2 and H3K9me3 demethylation, respectively. Furthermore, the down regulation of Oct4 during differentiation favors decreased Jmjd1a and Jmjd2c transcription, facilitating the incorporation of H3K9me2 and H3K9me3 and the epigenetic silencing of pluripotency-associated genes. Thus, histone demethylases play a key function in ES cell pluripotency maintenance and differentiation.

Another relevant aspect of ES cell epigenetics is the incorporation of histone variants. The histone variant H3.3 interacts with active and repressed genes in ES cells, in a HIRA-dependent manner. HIRA is a histone chaperone specific for histone H3.3 that mediates replication-independent nucleosomes assembly and appears to limit ES cell differentiation, suggesting that indeed H3.3 might influence the ES cell status.

DNA must be nucleosome free or “naked” while it is replicated with −250 bp or more of naked DNA trailing the replication fork. ATP-dependent chromatin-remodeling complexes regulate gene expression via moving, abrogating or restructuring nucleosomes. Energy from ATP allows these remodeling complexes to slide, twist or loop nucleosomes along the DNA, remove histones from DNA or swap variants to produce nucleosome-free regions of DNA for gene activation.

The ATP-dependent chromatin remodeling complexes are multiprotein complexes of variable compositions. Using energy from ATP hydrolysis, they relocate nucleosomes through sliding mechanisms and nucleosome eviction, induce changes in nucleosomes conformation and favor the interchange of canonical histones by histone variants. By these activities, chromatin-remodeling complexes contribute to gene expression activation or repression and label defined sectors of the genome through the incorporation of histone variants. ATP-dependent chromatin remodeling complexes are mainly grouped in the SWI/SNF, ISWI, CHD and INO80 families.

In comparison and importantly to the function of the TH2A/B histone variants, the chromatin remodeling complexes (CRCs) disrupt nucleosome—DNA contacts, move nucleosomes along DNA, and remove or exchange nucleosomes. In one embodiment, it is proposed that these variants, along with functional roles of other histone modifications previously unknown or classified as transient, or perhaps more directly as masking variants, are utilized to preserve the beneficial haploid genetic crossover of the parents during meiotic division and throughout early embryogenesis. These variant modifications act as first a shielding mechanism, and later as a conserved marker for the original modifications created during genomic crossover. They act across the spectrum of the genome, not as site specific regulators. Thus, it conserves lineage, but does not affect the “hardwired” genetic processes of core transcription factor regulation, but instead influences “individual” epigenetic modifications responsible for creating the individual. So, CRCs and histone variants serve to ensure DNA chromatin access to proteins that need to interact with DNA or histones directly during early development, or in the present disclosure, in the process of creating pre-embryonic cells.

Nucleoplasmin (NPML) is also involved in DNA replication, recombination, transcription and repair. NPML binds sperm nuclear basic proteins (SNBPs), including protamines, protamine-like type and histone types as part of the transition proteins (TPs). There are acidic regions throughout NPML (A1, A2, A3) that allow for charge neutralization by mimicking DNA, allowing for chromatin relaxation of specific regions and the ability for histone variants to enact a pivotal role in early formation, without permanent modifications. While nucleoplasmin promotes decondensation and remodeling of paternal chromatin following fertilization by exchanging SNBPs for histones, chromatin remodeling through phosphorylation of nucleoplasmin enhances H2A/H2B nucleosome assembly. It is also possible for NPML to assemble nucleosomes and decondense sperm DNA and activation of oocyte specific genes, allowing for multiple routes to ensure a primed environment. In some embodiments, the bivalent and conserved marks and CpG islands are of deep value. Likewise, the variants of histones in the early developmental phase target transcription sites and like the islands are primed with either a variant like H2AZ or H2A Lap1 on adjacent nucleosomes, or TH2B. One preserves the open structure while the other lends transcriptional on/off balance. This includes acting in place of a number of core transcription factors, promoting expression of Oct4, creating the self-replicating loop discussed in the CpG islands. CHD1 catalyzes the ATP-dependent transfer of histones from the NAP1 chaperone to the DNA, creating evenly spaced nucleosomes. Chromatin is destroyed, then reassembled during DNA replication and transcription through chromatin assembly factors, histone chaperones, HSPs and histone variants are likewise responsible for double-strand DNA repair and elongation. Histone chaperone Anti-silencing Function 1 (Asf1) is able to directly deposit histones H3 and H4 onto newly replicated DNA, while genome stability and preservation of specialized chromatin structures are preserved by chromatin assembly factor 1 (CAF-1) and Rtt106 with help from HSPs to position new histones on replicating DNA forming the nucleosomes. The supply of parental and newly formed histones at the replications fork is moderated via a histone acceptor/donor, Asf1-(H3-H4)-MCM2-7 intermediate. There are two crotonylated sites, by (H4K77) and (H3K122) on opposite sides of the nucleosome, which control electrostatic charges with the DNA backbone, which basically negates the positive charge, meaning that like the CpG islands another region of transcription can be easily modified to either express or silence transcription. While these mechanisms are highly conserved in normal oogenesis/spermatogenesis and embryogenesis, in one embodiment of this disclosure, they are initiated via the mechanistic application of massive environmental and chemical changes to induce an emergency state in the DNA. While these modifiers are conserved within the confines of a somatic cell, they are nascent to this process, as they are being recruited through a special set of circumstances and environmental freedom that was present only during fertilization and pre-embryonic development. These are transient protectors via histone variants preserved to enact specifically on regions of DNA from the seminal point of fertilization and genetic crossover. Thus, these are present in both the male sperm as well as the female oocyte, and further, this now conserved diploid set and comingled variants are present in the somatic cell undergoing the protocol, re-creating that pre-embryonic pattern embedded within the DNA to undergo totipotent renewal. In some embodiments, the addition of fibroblast growth factor (bFGF) activates the P13K which interacts with Akt thus boosting levels of Oct3/4, Nanog and Sox2, which in turn initiates the Ras-Raf-MEK-ERK pathway.

Several chaperones from the nucleophosmin/nucleoplasmin family are able to store histones and later transfer them when appropriate. When the remodeling of the histone and chromatin environment is viewed globally, the mechanisms of the present disclosure to create totipotent cells exist in a set of evolutionary scattered parcels. However, by performing the steps in the methods and protocols of the disclosure, one of skill in the art is able to initiate these dormant relics and through redirection of existing pathways like the heat shock family, create an environment and set of parameters that allows new function, or rewiring of these parcels to enact a global change and construction of newly formed pre-embryonic cells.

In one embodiment, the disclosed method of preparing conditioned stem cells creates joint synergies of initialized cascades, external energy sources, environmental pressures and the ATP-dependent chromatin-remodelers, histone chaperones, modifying enzymes and histone variants like TH2B, which orchestrate chromatin dynamics similarly to what occurs in early development. Through the protocol of the present disclosure, and the electromagnetic signaling capacity of the DNA and cellular system, histones undergo a spectrum of reversible posttranslational modifications, including acetylation, phosphorylation, methylation, ubiquitylation, and transient masking via histone variants and enzymatic bluffs. These modifications promote the recruitment of specific regulatory factors to create a dynamic euchromatin environment convenient for large-scale exchange of histones and formation of newly constructed totipotent cells.

This presents a model in conflict with stochastic varieties circulating in literature, as an immortal germ-like cell as well as the native imprinting pre-exists within every cell in all of the germ layers and the imprinting at critical gene promoter sites allows for the lineage specific epigenetic modifications according to cell-to-cell and environmental cues for somatic lines. In one embodiment, during the cellular remodeling phase of the protocol of the present disclosure, these “germ cell” bivalent markers are targeted by modification and repair enzymes to reinforce them and roll the DNA back to its earliest and most differentiation potential state—female/male totipotent germ cells, thus, conferring a more plastic environment for preservation/survival of the host DNA. It is in fact this plasticity of harboring this bivalent totipotent chromatin imprint that allows for the gamete to produce a zygote with proper gene regulation capacity to produce the desired lineage specific cells and tissues for development.

The molecular structure for a totipotent structure is hard wired into every cell. In one embodiment of the present disclosure, a carefully orchestrated catalyst of environmental signals to trigger surface and internal cell responses, initiation of a cascade of transcription factors, expression of chromatin modifying enzymes and other histone related enzymes, in addition to the initiation of critical protective genes from the heat shock factor family, are required. The first step in the protocol driving this cellular change is to initiate the heat shock factor protein 1 (HSF) gene to prime the cell. While interaction of transcription factors, histone modifiers, and chromatin remodeling factors will ultimately control the conversion of the naked DNA, the entire complex of transcription and growth factors, enzymes, protein interactions, activation of promoters and the chaperone complexes required to manage this colossal set of interactions is required. Expression of certain enzymes or factors within the body would lead to cell death, while in vitro, this is quickly countered via opposing expression—this initiation of multiple factors with inhibitory and at the same time catalyst for growth factors is what allows for regeneration, not ectopic or nuclear reprogramming.

Without wishing to be bound by any one theory, the initiation cascade of multiple heat shock proteins, including cold shock proteins, may be the primary conditional element for initiating the primary gene required at a specific time, and in turn, this gene activation produces the stepwise production of various signals that produce additional derivative transcriptions and activations. The initiation, blocking, repair and signaling that ultimately affects the nuclear DNA is fostered through this process with the help of heat shock proteins (HSPs). In this disclosure, numerous methods are introduced to amplify certain constrained elements of the cellular response and to aid in either the disruption of bonds or in enforcing others.

### Frequency Portion of Cavitation

Direct calculation of the partial atomic charges in molecules is applied to the nucleic acid bases. Inclusion of the published w-technique for the calculation of derived pi charges is used for highly polar systems. The partial atomic charges for cytosine, thymine, guanine and adenine (as the 1-methyl and 9-methyl forms) are compared with values calculated by a variety of molecular orbital and empirical outlines. The electrostatic contribution to the Watson-Crick base pair interaction energies are calculated using these partial atomic charges. From these calculations predictions down to an angstrom (0.1 nm) are made on the relative stability of base tautomers, identifying preferred protonation site within a base molecule, based on their electronic structure and the interaction energies between the various base pairs. Additional calculations of base pair monopole-monopole, monopole-induced dipole and dispersion energies, as well as interactions within polyatomic molecules illustrating electrostatic interactions in base pairing.

Electrostatic potentials to fit a partial atomic charge model for atom and united atom models. Empirical calculations, molecular dynamics and mechanics of the interactions between DNA and modifying elements use potential functions to describe the various bond stretching, angle bending, polarization terms, and theoretical “anchor points”, can be further compared and parameterized using known experimental data for smaller molecules involving polar molecules such as peptides and nucleotides.

For the DNA bases assignment of partial atomic charges can vary substantially between different sources due to configuration, thus individual sample calculations include the electrostatic contribution in these hydrogen bonded systems. Again, relevant calculations performed on amino groups and illustrative global data points are generated from each level, including an empirical scatter to derive the partial atomic charges, orbital electronegativities, with atom polarizabilities to calculate partial atomic charges via one, two and three bond effects extended to cover general pi systems for polar nucleic acid bases, the molecular charge distribution, the dipole moment of a molecule provides a highly accurate charge map.

The assumption that the molecular charge distribution can be represented by a series of partial atomic charges located on the individual atoms has been used repeatedly in industry applications for bio-engineering of materials. Dipole moments across methyl substitution has little or no effect for Uracil and Thymine since tautomerism is eliminated on N methyl substitution, the bases already have a calculated set of dipole moments. Calculated dipole moments In 1,7,7-trimethyl and 1,5,7,7-tetramethyl cytosine and two sets of calculations for dipole moments are used for coplanar and orthogonal orientation of the NMe2 with a ring are described and treated as a partial charge model to a quantum mechanical electrostatic potential. These are variably collected and transformed into targets for the electrostatic base pair interaction energies in kcal/mol which include for various methods.

Epigenetics refers to both heritable changes in gene activity and expression (in the progeny of cells or of individuals) and stable, long-term, alterations in the transcriptional potential of a cell that are not necessarily heritable. To include: (1) cytosine methylation; (2) post-translational modification of histone proteins and remodeling of chromatin; and (3) RNA-based mechanisms.

In addition to covalent modification of histones, chromatin structure is also controlled by families of enzymes that use the energy associated with ATP hydrolysis to effect changes in nucleosome arrangement or composition. These modifications are highly susceptible to ablation through directed frequencies changing the distribution of electrons forming the bonds created in the chemical post translational modifications. As such the entire structure atop of the DNA backbone can be systematically electrostatically neutralized, layer by layer without interrupting the underlying DNA structure.

Acetylation of lysine residues neutralizes their positively charged side chains, reducing the strength of the binding of histone tails to negatively charged DNA, ‘opening’ the chromatin structure and facilitating transcription and/or exposing DNA-binding sites. In a similar fashion, engineering targeted frequency disruptions allows an external manipulation of methyl sites, opening chromatin and/or removing entirely the post translation modifications, restoring a pristine DNA backbone. In biology, charge neutralization is thought to reduce affinity between histones and DNA, opening access to DNA for transcription factors and polymerases, and therefore enhancing transcription. Acetylation of specific lysine residues in histone tails is associated with gene activation. Lysine acetylation, catalyzed by histone acetyltransferases (HATs), neutralizes the positive charge on the lysine residues. It is likely you are beginning to see the correlation of magnetic-like properties. These associations between amino acids, through proteins, exist as combined and reinforced entities. The DNA structure likewise follows this same magnetic principle, further using this characterization through positive and negative attraction to allow geometry conformations, wrapping of histones in a solenoid fashion and then attracting and bonding with chemical assistance, modulators to cover or expose areas of open DNA (not wrapped and closed off via histone) to perform as the epigenetic workforce.

In summary, the covalent modification status of histone proteins, together with nucleosome composition and arrangement, comprises an epigenetic layer of information that facilitates or inhibits gene expression—properly calculating the electromagnetic or electrostatic charges of the histones, chromatin and modified sites, a directed outside source of energy can eliminate the secondary construct atop DNA entirely, or partially, through inactivation of the chemically bonded sites. It is similar in fashion to the natural chemical method employed within a cell, the acetylation event converts the positively charged amine group on the side chain into a neutral amide linkage. This removes the positive charge, thus loosening the DNA from the histone.

DNA demethylation is necessary for the reactivation of silenced genes, and in ‘cleaning the genomic slate’ during embryonic development. The literal ablation of all post translation epigenetic modifications through our engineered solution is what allows the generation of the powerful cunctipotent cell, which is the only naturally occurring cellular structure capable of producing every cell type, including germinal cells and allows for germ layer precursor cells to further differentiate into any cell.

Histone acetylation is the most widely studied epigenetic protein modification. Acetylation of specific lysine residues in histone tails is associated with gene activation. Lysine acetylation, catalyzed by histone acetyltransferases (HATs), neutralizes the positive charge on the lysine residues. This charge neutralization is thought to reduce affinity between histones and DNA, opening access to DNA for transcription factors and polymerases, and therefore enhancing transcription.

The negative charge on cytosine is stabilized by interaction with a glutamate residue. Histones have many arginine and lysine amino acids that easily bind to the negatively charged DNA, DNA is highly negatively charged because of the phosphate group of each nucleotide is negatively charged.

Histones are divided into two groups: Core histones and Linker histones

| Modification | R-group | Charge | Effect |
|---|---|---|---|
| Methylation | R-CH3 | Neutral | Increases packing |
| Acetylation | R-COCH3 | Negative | Decreases packing |
| Phosphorylation | R-PO4 | Negative | Decreases packing |

Histones are positively charged molecules, and the addition of methyl groups (methylation) makes them more hydrophobic thus tighter bonding, and increasing histone methylation will cause the hi stones to pack even more tightly than usual. Acetylation (adding an acetyl group) and phosphorylation (adding a phosphate group) make the hi stones more negatively charged because acetyl and phosphoryl groups are negative. Histones more negatively charged, their grip on DNA will be much looser because DNA is also negatively charged. Similar charges (negative and negative) repel one another. This is the core of our engineered solution. The nuclear forces that are the building blocks of everything persist even at the macro level. The chemical basis of these modifications are governed by the interactions of the nuclear particles and thus these modifications are subject to external forces utilizing targeted forces to interrupt their molecular configuration, thus “unwinding” the modifications. The extreme heat generated through cavitation and the denaturing temperature that is included during the engineering protocol break down these loosened structures making the component pieces available for the newly created cellular membrane and naked DNA to use in development.

Double-stranded DNA consists of two polynucleotide chains whose nitrogenous bases are connected by hydrogen bonds. Within this arrangement, each strand mirrors the other because of the anti-parallel orientation of the sugar-phosphate backbones, as well as the complementary nature of the A-T and C-G base pairing. The sharing of electron pairs in carbon-carbon covalent bonds may be as a single bond or with double bonds. Single bonds have complete freedom of rotation, while double bonds are shorter and do not allow free rotation. The type of covalent bond is therefore important for electrical properties such as polarization and relaxation time. An electron revolving around its nucleus may be considered as a rotating electrical dipole. Such a rotating dipole induces dipoles in neighboring atoms. Van der Waals forces are dipole-dipole attractive forces between such atoms. The forces are weak, and fall with the sixth power of the interatomic distance. Many organic molecules form aggregates (heterogeneous mass of parts or particles) held together by van der Waals forces.

Hydrogen (63% of the human body's number of atoms), oxygen (25%), carbon (9%) and nitrogen (1.4%) are the four most abundant atoms of the human body. They are all able to form covalent bonds based on the sharing of electron pairs by two atoms with unpaired electrons in their outer shells. Most biomolecules are compounds of carbon, because of the bonding versatility of this element. Nearly all the solid matter of cells is in the form of: water, proteins, carbohydrates and lipids. The living cell must contain and be surrounded by aqueous electrolytes. In human blood the most important cations are: H, Na, K, Ca, Mg; and anions: HCO 3, Cl, protein, HPO 4, SO 4. Protein in the blood is considered a negative ion, Electrolytes inside/outside cells and both intra-cause an electrolytic conductivity of the order of 1 S/m. Proteins are the most abundant macromolecules in living cells, and 65% of the protein mass of the human body is intracellular.

Proteins are the molecular authors through which genetic information is expressed. Proteins are constructed from ˜20 amino acids, joined by covalent bonds. All 20 amino acids get an R group. At pH 7 all amino acids effectively polar. Peptides are small groups of amino acids, and polymers are an iterative layer of peptides forming proteins. Proteins are unique because each has its own amino acid sequence.

Now, a denatured protein always loses its characteristic biological activities, and the electric properties are completely changed. A DNA molecule consists of two polynucleotide chains (helices). The two chains intertwine with a fixed pitch of 3.4 nm. Two types of base pairs bridge the two helices at a fixed distance of 0.34 nm. The phosphate groups in the nucleotide chains carry negative electric charges in water. An electric double layer covers the wetted outer cell membrane surface. The total cell has a net charge revealed by its electrophoretic mobility. The cell membrane capacitance with the thickness of about 7 nm is of the order of 1 F/cm 2. The cell membrane has a frequency independent capacitance. If the potential difference is increased by ˜150 mV, the membrane breaks down. In commercial applications like electroporation, higher frequencies allow for membrane capacitance to let AC current pass. The membrane effect disappears, current is guided by conditions of cells ionic conductivity.

In a frequency range 0.1-10 MHz, the phase angle is maximum (the Maxwell-Wagner/)-dispersion range for the dielectric interfaces. The dipolar dispersion of the proteins also appears in this frequency range, and it is still active above 100 MHz. This is the y-dispersion range extending all the way up to the single Debye characteristic frequency of water.

With an applied electric field, the electron cloud is displaced and a dipole moment is generated an applied electric field induces dipole moments in a dielectric. Such a displacement of charges generally generates a dimensional change in the material. This is called electrostriction.

In a similar manner, mechanical stress changes the dimensions of the material, does not result in an electrical polarization of the material unless there are crystalline structures and they then, because most materials generate an internal polarization when mechanically deformed. These materials are called piezoelectric with a direct conversion from mechanical to electrical energy. It is claimed here that many of the protein structures contained along the chromatin structure on the DNA backbone share characteristics similar enough to crystalline structures to form piezoelectric requirements.

T75 at confluency are 8.4×10⁶ cells. Cultured cells (10⁷ cells) yield ˜30-70 micro grams DNA. Mitochondria (10 mg tissue, 10⁷ cells) 1-10 micro grams. The size of diploid human genome is around 6 billion bp. Average size of a nucleotide bp=660 g/mol. DNA from a single cell=660×6 billion=3.96 pico gram (pg). About 3 billion in the haploid human genome, you may calculate as follows: 3×10⁹ bp×2 (diploid)×660 (AVGed MW of 1 bp)×1.67×10⁻¹² pg (“weight in dalton”)=6.6 pg/diploid primary cell. Human cell membrane is only about 7 nm.

### Epinul Cells

It is fundamental to point out the nature in which the following list of engineered cell products are created. While individual forms of many of these are produced inside of either a male/female, the cell products created through the methods described herein are not naturally occurring and have been designed and engineered from a somatic cell source from a host. These are genuinely a synthetic re-creation of the earliest and most plastic cell sources, cunctipotent cells responsible for the rise of an organism through the natural process of development. The starting host cells are terminally differentiated cells.

The only known mechanism for converting somatic cells presently is through exogenous gene transfer to stimulate key transcription sites with the genes to attempt to mimic early cell expression, thus leading toward a more “stem-like” cell. iPSC stands as the leading technique, awarded a Nobel Prize and currently the only way to generate pluripotent stem cells from differentiated cells.

The actual equivalency of iPSC cells to embryonic stem cells has been questioned, in relation to gene expression, methylation patterns and most important functional ability. Numerous groups have found differences in gene expression, DNA methylation and differentiation propensity between iPS cells and ES cells. Further, it has been suggested that induced pluripotent stem cells are not reprogrammed to the same extent that is observed in embryonic stem cells following nuclear transfer. Tests on numerous differentiated lines, ranging from cardiac, to neural, muscle and functional cells like insulin secreting beta cells. In each of the many examples, gene expression, behavior and physical robustness of the cells were found to be grossly lacking. No clinical trials presently use iPSC or ESC as the source cell for therapeutics.

Embryonic stem cells are not a natural cell type found in nature—these cells (for which iPSC are measured) are created from isolating a single group of cells inside of a fertilized blastocyst. As such, many of the developmental instructions, cues for activation of numerous genes and critical cell-to-cell programming are forever lost. This is readily validated and documented in scientific and medical research and publications. These deficiencies include lost expression of more than two dozen genes found within the normally developing blastocyst. In both cases, tremendous sacrifices are being made to produce cell platforms that were designed to be the “holy grail” of therapeutic and regenerative medicine. ESL's are only created through the destruction of a healthy blastocyst—therefore, there can never be an “autologous” platform generated for any individual with this technique. Outside of the epigenetic erasure protocol, engineering a genuine clone or autologous totipotent cell line for an individual does not exist.

Where iPSC starts with somatic cells from a specific host, the process of corrupting the genome to force transcription expression again translates to an impossibility of creating a genuine “autologous” cell platform. Similar to ESC, as well as thoroughly documented bragging rights, they share “almost” identical transcription and active gene profiles. The problem for therapeutics and regenerative medicine; these cells either missed out completely on the earliest forms of programming through the embryonic phase, or have been teased into pseudo pluripotency with a host of post translational modifications from previous differentiation in the form of methylation, acetylation, etc. The most grievous and distinctive element that sets both platforms apart is the lack of more than two dozen genes that are active and critical to development, present inside a healthy blastocyst are notably missing from each of these cell products.

Epinul cells. Methylation of DNA is an essential epigenetic control mechanism in mammals. During embryonic development, cells are directed toward their future lineages, and DNA methylation poses a fundamental epigenetic barrier that guides and restricts differentiation and prevents regression into an undifferentiated state. DNA methylation also plays an important role in sex chromosome dosage compensation, the repression of retrotransposons that threaten genome integrity, the maintenance of genome stability, and the coordinated expression of imprinted genes. However, DNA methylation marks must be globally removed to allow for sexual reproduction and the adoption of the specialized, hypomethylated epigenome of the primordial germ cell and the preimplantation embryo. Recent technological advances in genome-wide DNA methylation analysis and the functional description of novel enzymatic DNA demethylation pathways have provided significant insights into the molecular processes that prepare the mammalian embryo for normal development. The developmental pathway provides for Cunctipotent Primordial Cell—Primordial Germ Cell—Oocyte.

An oocyte is a female germ cell involved in reproduction. It is one of the largest cells in the body and develops in the ovarian follicle, a specialized unit of the ovary, during the process of oogenesis/folliculogenesis in the cortex. The process of oogenesis starts in the fetal ovaries with the development of oogonia from primordial germ cells (PGCs). Each oogonium in the fetal ovaries divides and enters the initial stage of meiosis (meiosis I) to become the diploid primary oocyte. (This is a similar process in terms of developmental progress with our engineered cells. However, the diploidy of the cell in this case is already a genetic cross—autologous—as it is generated from the host.)

As opposed to mice, in humans, most of the genome-wide demethylation is complete by the 2-cell stage. Paternal genomic demethylation happens much faster than in the maternal genome. The traditional promoter methylation/gene expression relationship grows during early embryonic development. peaking at post-implantation. Active genes marked by H3K4me3 in their promoters are not methylated in pluripotent embryonic stem cells. Zygotes are formed as a direct result of the DNA needing to demethylate.

Blastocyst and stem cells derived therefrom are fully competent clonal colonies of individual pluripotent cells have undergone the transformative steps of division from a single (synthetically fertilized) zygote, through morula and blastocyst development to the hatched peri-implantation embryo stage. They express markers found within the healthy blastocyst and imperatively contain populations of cells from the multiple cellular layers of the blastocyst, which together are capable of producing all of the cells, tissues and organs making up an individual.

Blastomeres—Progenitor and derived cells directly from protocol or differentiated from above protocoled cell types.

In the process of fertilization, the lucky few sperm who reached the egg in the Fallopian tube surround it and begin competing for entrance. The head of each sperm, the acrosome, releases enzymes that begin to break down the outer, jelly-like layer of the egg's membrane, trying to penetrate the egg. Once a single sperm has penetrated, the cell membrane of the egg changes its electrical characteristics. This electrical signal causes small sacs just beneath the membrane (cortical granules) to dump their contents into the space surrounding the egg. The contents swell, pushing the other sperm far away from the egg in a process called cortical reaction. The cortical reaction ensures that only one sperm fertilizes the egg. The other sperm die within 48 hours.

The fertilized egg is now called a zygote. The depolarization caused by sperm penetration results in one last round of division in the egg's nucleus, forming a pronucleus containing only one set of genetic information. The pronucleus from the egg merges with the nucleus from the sperm. Once the two pronuclei merge, cell division begins immediately. The act of fusion of the sperm and the method of approach, entry and eventual fusion of the male and female nuclei constitutes the mechanism of fertilization. This is believed to take place in the following stages. 1. Movement of the sperm towards the egg. 2. Capacitation and contact. 3. Penetration of sperm into ovum. 4. Cortical reaction. 5. Activation of the ovum. 6. Fusion of male and female pronuclei (amphimixis).

Since age has a strong effect on DNA methylation levels on tens of thousands of CpG sites, one can define a highly accurate biological clock (referred to as epigenetic clock or DNA methylation age) in humans and chimpanzees. DNA methylation marks must be globally removed to allow for sexual reproduction and the adoption of the specialized, hypomethylated epigenome of the primordial germ cell and the preimplantation embryo.

However, these epigenetic barriers also pose a major challenge to sexual reproduction, where preparation for the next generation requires a reset of the (epi)genome to a basic, totipotent state. Particularly in mammals (where germ cells are not defined at fertilization but rather arise from later embryonic tissues), resetting the epigenome is of great importance.

In contrast to the enzymatically controlled, straightforward methylating mechanism, a direct DNA demethylase capable of breaking carbon-carbon bonds has not yet been identified commercially. Furthermore, this mechanism does not allow locus-specific, but only global, removal of DNA methylation marks. Indeed, recent studies indicate that passive dilution of methylation might well be sufficient for global demethylation, providing an energetically more plausible, robust mechanism. (breaking down large enough portions confers a global restructuring) DNA methylation patterns in vivo are primarily informed and shaped by DNA sequence, classical CGI like sequences in large genomic regions of DNA can be interpreted by evolutionarily conserved mechanisms, to protect these sequences from DNA methylation and to shape the epigenome during development.

The following examples are presented in order to more fully illustrate the various embodiments of the disclosure. They should in no way be construed, however, as limiting the scope of the disclosure.

### EXAMPLES

### Example 1: Production of Synthetic Engineered Totipotent Cells, i.e., Cunctipotent Cells (“SEC”) from Somatic Cells

This study demonstrates the results from a novel method for the creation of cunctipotent cell lines for personalized therapeutics, as well as conventional uses currently filled by iPSC and ESC methods. Directed applications of electromechanical, electromagnetic, frequency shifting and ablation, extreme heat through cavitation and environmental factors associated with excitation of specific genes; and enhancement of this catalyst through engineered and specifically timed alterations to environment, drive the genome of somatic cells to full pluripotency. The methods are performed in the absence of exogenous transcription factors, vectors or genetic material. The ability to produce cunctipotent primary cells with global epigenetic erasure without impact on, or genetically altering/reprogramming adult cells, provides a means for patient-specific stem-cell-based therapies.

Pluripotent embryonic stem cells; and induced pluripotent stem cells are known and used in the art, see for example Thomson, J. A. et al. (1998). Embryonic stem cell lines derived from human blastocysts. Science, 282(5391): 1145-1147; and Takahashi, K., & Yamanaka, S. (2006). Induction of pluripotent stem cells from mouse embryonic and adult fibroblast cultures by defined factors. Cell, 126(4): 663-676. While these advances have led to a useful tool in diagnostics, there are still differences in electrophysiology properties between human ES cells and human iPS cells (Jiang, P. et al. (2010). Electrophysiological properties of human induced pluripotent stem cells. American Journal of Physiology-Cell Physiology, 298(3): C486-C495). Evidence shows that the process of creating iPS cells has a number of drawbacks, among other aspects: foreign genes were silenced or removed after reprogramming; reprogramming occurred at low efficiency; the process left residual vector sequences; the process required tedious steps and did not produce stem cells genetically identical to the host.

Using the methods provided herein, normal human MRHF—Human Foreskin Fibroblast (Quidel) and McCoy—Mouse Fibroblast (Quidel) cells were engineered to cunctipotentcy. The engineered synthetic cells precisely duplicate human and mouse embryonic stem-cell-like colonies in morphology and gene expression, while also generating a new and novel source of primary cunctipotent pre-germinal cells that are the earliest and most diverse cell in nature, a cell type not ever commercially produced. As well, oocyte and competent diploid zygotes, and individual satellite blastomeres are produced: they were alkaline phosphatase-positive; human cells expressed OCT3/4, TRA-1 to 60 and TRA-1 to 81 proteins; while mouse cells were positive for SSEA1, Sox2, Nanog and OCT3/4. RT-PCR performed on human cells showed that the engineered cells expressed genes analogous to embryonic stem cells. These results suggest that the protocol utilized to produce the SEC cells may lead to the development of novel therapies. The creation of cell lines from an individual or a patient with disease, genetic disorders or for purposes such as blood and tissue banking may be possible by the reconditioning of somatic cells into a cunctipotent state. These methods allow for the immediate creation of autologous and genetically identical/compatible pre-embryonic cunctipotent, totipotent or stem cells from an individual's adult human cells for therapeutic purposes.

The efficiency of the present methods provides significant benefits. The protocol can be completed in 1.5-2 hours. The methods are scalable, where sample size can vary considerably before there is any noticeable impact in the end product. The engineered cells are robust and durable. Cost of materials and manpower required to perform the protocol is low, and the protocol can be performed in an automated and self-contained system. A high percentage of starting cells are engineered to cunctipotentcy, for example at least about 20%, at least about 30%, at least about 50% or more. Starting cells can also be engineered to primordial germ cells, embryos, totipotent colonies, etc. Patient-specific stem cell lines can be generated to study various disease mechanisms, offer valuable tools for drug discovery, and provide a robust platform to design customized patient-specific stem cell therapies with economic feasibility.

Normal human foreskin fibroblasts (MRHF) and mouse fibroblasts (McCoy) were received in 1 mL frozen vials, thawed and grown in engineered media as follows. Pre-protocol (starting) human and mouse cells were cultured in Dulbecco's modified Eagle's minimal essential medium (DMEM) supplemented with 10-15% refined fetal calf serum, 0.1 mM nonessential amino acids. Post protocol (engineered) colonies were cultured in Dulbecco's modified Eagle's minimal essential medium (DMEM) supplemented with 10˜15% refined fetal calf serum, 0.1 mM nonessential amino acids, 0.1 mM β-mercaptoethanol, and 100 U/mL leukemia inhibitory factor (LIF).

The engineered stem cells (SEC cells) grew in culture flask coated with 0.1% gelatin and were maintained at less than 60% confluency to keep an undifferentiated phenotype. Once the cells reached 60% confluency, passage of cell was conducted at a 1:8 subculture ratio until testing at which point the cells were allowed to reach ˜80% confluency.

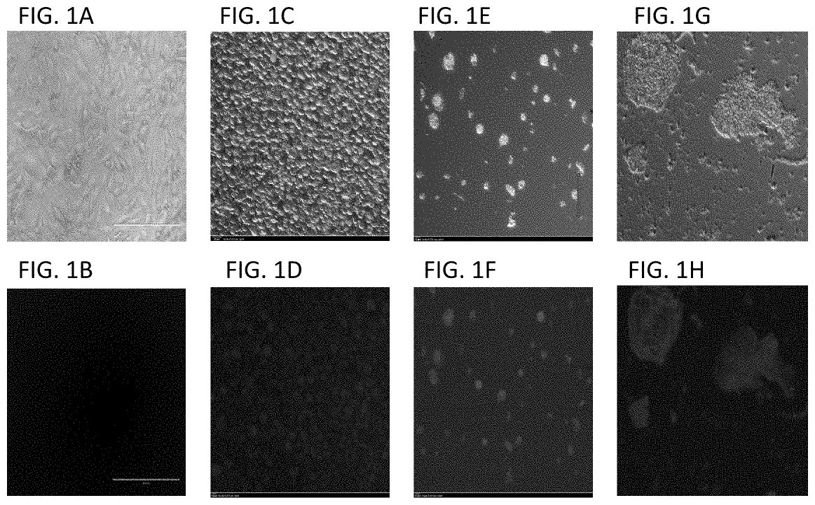

After 5 days of growth, the cells were ˜70% confluent, at which time initial images of the cells were taken to establish health and morphological basis. The cells from each human and mouse line were kept separate and isolated and all experiments were performed on a single species at a time to prevent any cross contamination. The confluent cells were trypsinized after 5 d of growth and split at a 1:20 ratio to allow for 1 mL cell samples at ˜2×10⁴ cells/sample. All tests and controls were set up in triplicate to allow for a minimum of two complete control sets per protocol. Negative controls included original trypsinized cells left untreated and reseeded, an individual sample for each antibody test and a negative control with conditioning elements utilized during the protocol were reseeded into 6-well plates and placed in incubation. CD-3 was used a positive control for the fibroblasts. Post protocol cells were reseeded and images were taken at 1 hr, 6 hrs, 12 hrs, 24 hrs, 36 hrs and 72 hrs to document changes and track morphological characteristics (see FIG. 1). A comparison with control cells as early as 1 hr after protocol showed significant structural and morphological changes in the cells, and by 12 hr post-protocol distinct colonies were readily identified, exhibiting morphology comparable to human embryonic stem (hES) cells/mouse embryonic stem cells (mES) (FIG. 1C). Colonies displayed the morphology characteristic for undifferentiated hESCs/mESCs (i.e., large, compact, multicellular colonies of cells with a high nucleus-to-cytoplasm ratio).

The SEC protocol treated cultures were passaged by trypsin and mechanical dissociation. As a result of the rapid formation of the SEC colonies, cell adherence was a factor and a special plate coating (Matrigel) was recommended. Upon extended in vitro culture, without passaging and the addition of LIF (Leukemia Inhibitory Factor) and bFGF for human cells to the growth media, the colonies differentiated spontaneously and generated heterogeneous populations of cells with a variety of morphologies. From a 1 mL sample with ˜2×10⁴ MRHF or McCoy cells, each well contained an average of 150 colonies varying in size and shape were observed and sample regions imaged. Colonies from one set of each protocol and the untreated negative control were stained with alkaline phosphatase. Positive indications were observed on large and small clusters in each well of the cells treated according to protocol, while a negative or no staining was exhibited by the control. Positive alkaline phosphatase staining is a phenotypic assessment of undifferentiated ES cells (FIG. 1 F).

To confirm and assess the cunctipotentcy as well as pluripotency of the engineered cells with consideration to characteristics of typical ES cells, tests were performed to measure stem cell marker expression following manufacturers' methods.

Cells were detached using trypsin and separated into 20 1-mL individual samples and reseeded for 24 hours. The media was drained and cells were washed with PBS and then fixed in 4% paraformaldehyde washed and permeabilized with 0.5% Triton X-100 in PBS— (Life Technologies) for 30 min and blocked with 10% FCS in PBS— for 1 h. Cells were incubated with primary antibodies in PBS, 1% FCS, and 0.5% Triton X-100 at 4° C. for 1 h. After washing 3 times, nuclei were counterstained with 4, 6-diamidino-2-phenylindole dilactate (DAPI; 1:10,000, Sigma). All cells were visualized using a fluorescence microscope (Axio Observer, Zeiss). After permeabilization and blocking, the cells were incubated for 20 hours at 4° C. with primary antibodies directed against the following antigens: SSEA-1, TRA-1-60, TRA-1-81, Oct3/4, SOX2, Nanog. Dapi and Hoescht were used for nuclei staining. The stained engineered protocol cultures were mounted and visually inspected in an inverted fluorescence microscope (Thermo EVOS fl imaging system). Immunofluorescence for the human ES cell specific proteins TRA-1 to 60 and TRA-1 to 81 were performed using an ES cell characterization kit (Millipore) and an OCT-3/4 primary antibody (BD Biosciences). Mouse ES cell specific markers SSEA1, OCT3/4, Nanog and SOX2 kit was used (BD Biosciences). Alkaline phosphatase staining for the phenotypic characterization of pluripotent cells was assessed using an Alkaline Phosphatase kit (Stemgent).

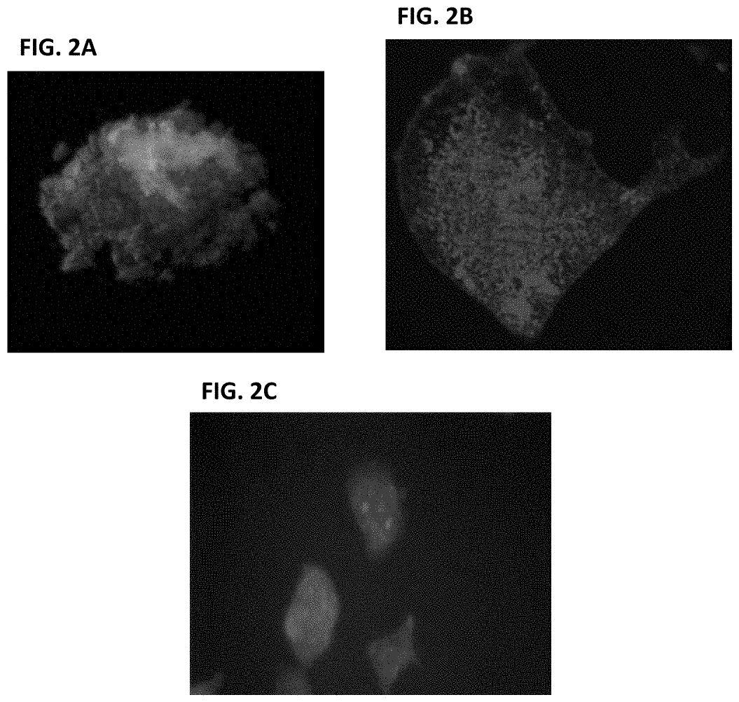

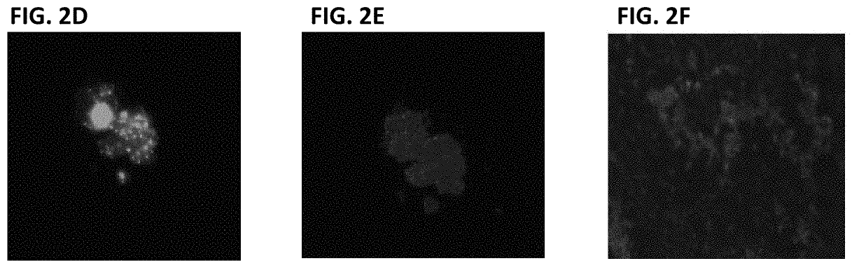

Immunofluorescence microscopy showed that the protocol engineered MRHF cells showed localization of OCT3/4, TRA-1 to 60 and TRA-1 to 81 proteins (FIG. 2A-FIG. 2C) and protocol engineered McCoy cells showed localization of OCT3/4, SSEA1 and SOX2 (FIG. 2D-FIG. 2F).

RNA was extracted from MRHF engineered protocol cells (36 hours post protocol) and from MRHF non-treated cells at 36 hours after passage (MRHF cells were obtained from Quidel) using an RNA isolation kit (Invitrogen). PCR for endogenous stem cell marker genes was performed using TaqDNA polymerase (Invitrogen) and a SuperScript III first-Strand Synthesis system (Invitrogen), in adherence to the manufacturer's instructions.

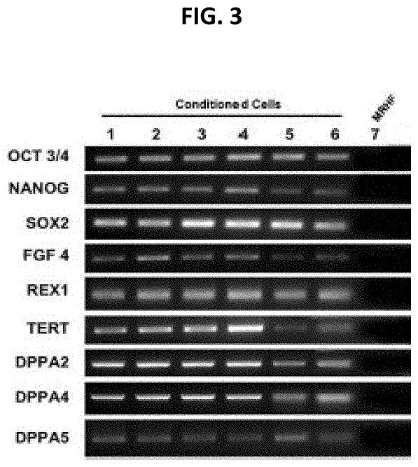

Reverse-transcriptase PCR (RT-PCR) analysis performed on MRHF cells confirmed that the protocol engineered cells expressed stem cell marker genes for OCT3/4, NANOG, SOX2, fibroblast growth factor (FGF)-4, reduced expression protein 1 (REX1), telomerase reverse transcriptase (hTERT), developmental pluripotency-associated (DPPA)-2, DPPA4 and DPPA5 (FIG. 3).

This technology has the potential to minimize the risks from use of viral vectors and genetic matching techniques currently utilized. The data in this study indicates that the creation of engineered stem cells from somatic cells is safe, fast and efficient. In view of the majority of existing in vitro cell reprogramming methodologies that generally achieve pluripotent (iPS) cell generation within 3 weeks with less than 1% of efficiency, in vivo reprogramming to pluripotency may offer very interesting alternatives.

### Example 2: Production of Blastocysts from Somatic Cells and Spontaneous Differentiation into Pluripotent Embryonic Stem Cells

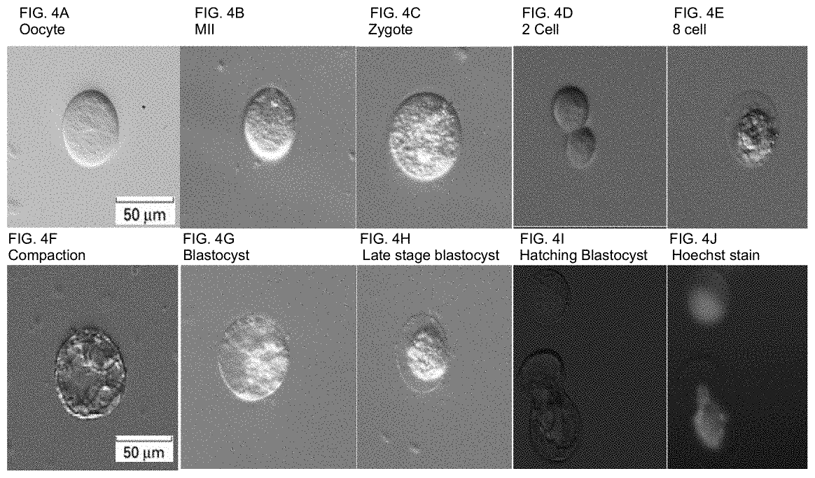

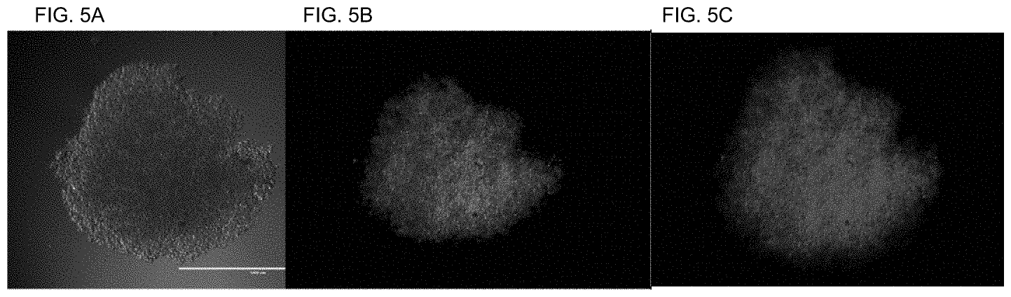

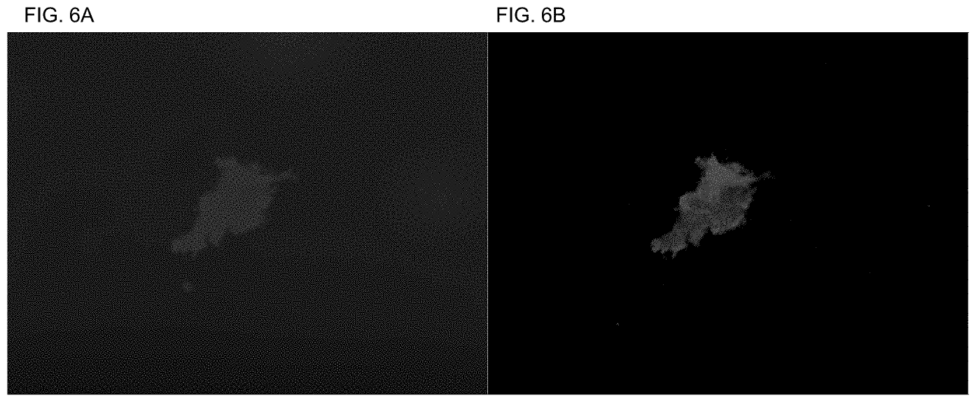

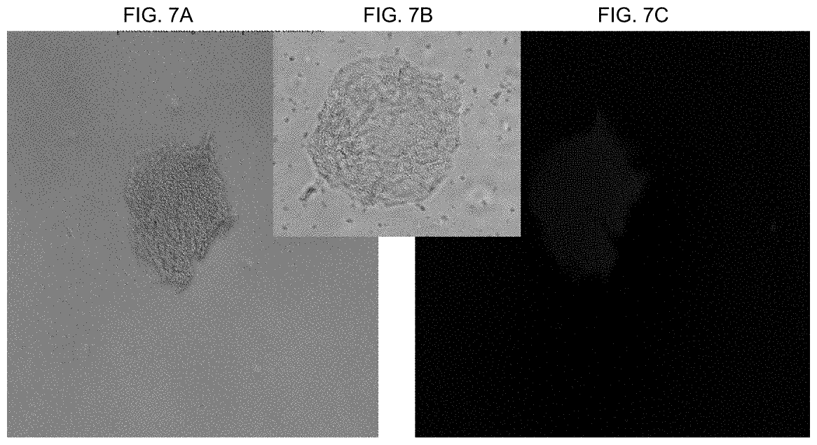

SEC cells, zygotes and blastocysts were formed 24-36 hours post protocol (see FIG. 4). Small flat pluripotent stem cell colonies tested positive for traditional markers of embryonic stem cells (FIG. 5, FIG. 6). The inner cell mass from blastocysts were separated by manually breaking open the membrane and allowing cells to seed in gelatin coated wells. Embryonic stem cell colonies form within 24 hours and continue to grow (FIG. 7). Cutting the stem cell colony into pieces and seeding in new wells results in continual growth and formation of new colonies, positive for traditional markers of embryonic stem cells.

Zygotes, morula, blastocyst and SEC cells were tested for traditional markers (nuclear Oct-4, cell surface protein SSEA-4, TRA 160, TRA 180, Nanog, Sox-2, Rex-1, and Tert) as well as E-cadherin, c-kit and for the SEC cells, the most prominent indicators of totipotency—SALL4, CDH1, LIN28B, SOX11, LEFTY1, PP1R9A, MYBL2, and HESRG and germline markers—PRDML (BLIMP1), PRDML4, and DPPA3 (STELLA).

Cells exhibited the characteristic features of natural oocytes, like germinal vesicle formation, extrusion of polar body, and formation of distinct zona pellucida.

### Example 3: Methods for Epigenetic Erasure and Engineering Cunctipotency

Heat, force, magnetism, electricity and cell signaling, enhanced by chemical reactions at specific stages are applied to a starting cell population to enact a cascade of transcription signaling. The steps are designed to impact specific families of factors, produce proteins, internal and external cell signaling and disruptive environmental pressures. In each step the initiation of the proceeding events within the cells and the environment are needed to enact further expression of regulatory networks, the induction of the HSP network and the process of remodeling and preparing the histone and chromatin of the nuclear DNA to enact a holistic preservation reaction—for purposes of this platform, this process is called Basic Biological Nuclear Preservation (BBNP) that results in epigenetic erasure.

The origins of the host will de-evolve into the most suitable range of cellular structures that will allow for repair, reconstitution, preservation and ultimately the ability to replicate. This process essentially sheds the epigenetic constructs of the chromatin and histone structure to allow for an open genome in a cell capable of cunctipotential and totipotential growth and differentiation. As such, the biological clock of these structures is reset and a reversion to the germ line, pre-embryonic state is achieved.

Cells grown in typical T75 flask to 80% confluency are taken from incubation, washed with 5 ml room temperature PBS and trypsinized with 5 ml 0.25% 37° C. trypsin, typically resulting in a starting cell population of 8.4×10⁶. This begins the process of softening the cell membranes, detaching the healthy cells from the substrate and inducing cold shock, recruiting specific CSP/HSP networks. Against convention of standard cell biology methods, scraping, and rough treatment of the cells throughout the process is integral to providing hostile conditions which drive the networks of reactions, damage portions of the cell sample and enact various mechanisms of survival and the early stages of the apoptotic process.

Cold media is added to produce a preferred range of working size and initial population of cells per sample. In this embodiment, a total 20 mL of cells, trypsin and media are separated into 2 ml aliquots, approximately ˜1×10⁶ per ml into 15 mL tubes for further treatment.

Individual solutions of ATP (100 μM/ml), EGF (2 μM/ml), Insulin transferrin selenium (20 μM/ml), I-ascorbic acid (0.5 μM/ml) and retinoic acid (0.01 μM/ml) are measured out prepared, then suspended in DMEM. For this sample size approximately 1 million cells will be added to the solution. A gradient of these diluted solutions is administered to individual 15 ml samples. Cavitation is then used to disrupt the cells in suspension.

In a manual protocol, ultrasonic energy is produced from the microtip of the sonicator generating intense frequencies and cavitation which is directed downward and outward from the vibrating tip. As the solution is being processed, the liquid is pushed in all directions. Based on the size and shape of the vessel, a 15 ml plastic conical tube used, a ¼″ microtip is used for 2 ml volume. (Calculations take into account the variance in work and magnetic properties from the center of the tube directly in contact to the edges. This in fact creates a part of the variable environment of disruption to cells, destruction of cells and the denaturing of proteins to “seed” the solution with desired factors and raw materials.)

The device is set to power: 700 watts—measure of energy per unit of time that is conveyed from the generator to the sonicated liquid measured in watts (W) or kilowatts (KW) delivered to the sonicated liquid via a unit (cm\hat{ }2) of the horn's radiating area.

Frequency: 19-21 kHz will give 19-21,000 vibration cycles per second. Amplitude: 37 dB.

The effectiveness of ultrasonic processing is directly related to the intensity, (amplitude and intensity have a direct relationship) homogeneity and size of the cavitation field created in the liquid, thus the “solution” properties are also calculated to determine calculations for the engineered epigenetic disruptions desired.

In this example, 15 ml conical tubes were filled with a solution of about 10⁶ starting cells, media and additives as stated above, and the final volume brought 2 ml with media. Sonication was performed in a series of 3 steps, beginning with sonication and cavitation via a probe inserted into the mixture, going from the top, nearly teaching the base and returning up and out of the solution. Total time for this calculated protocol is three 5 second bursts. Amplitudes are variable for different cell sources and densities—for this application, amplitude is set to 37, frequencies between 16 kHz and 20 kHz, with power 45 W producing 1,350 j of energy. Probe temperature was set to 65° C. for 17 seconds with pulses from 1-5, 7-11, 12-17 seconds (2 one second interrupts).

Cells were effectively disrupted and after sonication/cavitation, environmental pressure changes, desired shear, and secondary cavitation were achieved using a strong electric pipette with a narrow tip and larger body, such as a 5 ml. The sample was pulled in through the narrow opening as rapidly as possible, creating shear force, heat and constrictive pressure/atmospheric pressure in rapid, alternating fashion producing weaker forces generated from modulating waves. This was followed immediately by the sample re-expanding rapidly in the larger body of the tube, creating rapid expansion—as this process is repeated rapidly, pulling the sample into the tube and forcefully depressing it back into the 15 ml tube, microbubbles formed in the solution. These microbubbles create miniature thermal events when they rupture, enforcing changes on the cells, damaging others; and in the process initiating cellular HSP response, catalysis of early reactions, and aiding enzymatic process.

Optional additions to the protocol include, for example, addition of various inhibitors like valproic acid (VPA, an HDAC inhibitor), AZA (a DNMT inhibitor), butyrate (an HDAC inhibitor), CHIR99021 (a GSK3-β inhibitor) and PD0325901 (a MEK/ERK inhibitor) and other small molecules that have been shown to support ESC and iPSC in reprogramming, which however are used here for post-protocol development.

The inclusion of ATP in the media provides available energy required to rapidly respond to epigenetic erasure of methylated sites, conduct repair, and convert the cell to a primordial state.

Following the epigenetic erasure step, ITS (insulin transferrin selenium) and EGF (epidermal growth factor) diluted into DMEM were added to the sample and titrated through the sample to ensure maximal dispersion throughout the sample.

An additional fetal bovine serum (FBS to a final concentration of 15%) was added to each sample to saturate free amino acids into the sample environment added.

As the cascade of events are happening within the sample, initiation of gene transcription for the core transcriptional factors is assisted via multiple mechanisms initiated from the former steps. Additional cavitation/manual titration of the sample is required to bring about the destruction of more cells, damage to neighboring cells and full expression simultaneously of different pathways that are intertwined—the cascade of these pathways initially stimulate the production of proteins and signaling of pro-survival as well as proteins required from the apoptotic direction that will act as inhibitors and modulators to the process during the above described protocol. The combined over stimulation of these distinct and separate pathways cross pollinate to enact the express conditions required to stimulate pre-transcription networks responsible for engineering cunctipotent transcription within the nuclear and mitochondria! DNA and globally erasing methylation. Cavitation/titration is carried out until there is a slight change in color the sample. If using phenol red containing media, a slight pinkish hue will resolve after sufficient work is applied. Stop the process and allow the solution to settle and a rim of white foam to aggregate around the top.

In one embodiment, the cell sample is loaded into a device providing a series of predetermined acoustic waves of varying strengths in a cyclic manner to enact sheer forces, velocity and extreme pressure changes to the environment. The cavitation or ultrasound process generates the formation of micro-bubbles and the successive destruction of these bubbles causes localized and extremely small regions of extreme heat, sometimes nearing 5,000 degrees' kelvin. This creates monomers, initiates the highest order of heat shock response and initiation of the BBNP survival cascade.

In this embodiment, the color of the sample will change slightly as the process introduces micro-bubbles into the sample. After the solution has rested and is once again clear, an addition of 1 μl/ml of proteinase K is now added to act as a chromatin digest and cavitation/titration was resumed for a short duration. If manual, this is 3-5 pulls of the sample in and out of the pipette.

The samples were then immediately removed and placed into a heat bath at 65° C. for 2 minutes (This is specific to 2 ml samples in a 15 ml tube and depending upon the method used for cavitation.) The heat is sufficient to neutralize the proteinase and further denature free cell products in the environment, e.g. in reformation of lipid bi-layers, enzymes and cascade signaling molecules. This step again enforces the activation of HSP family and recruitment of additional chaperonins and co-activators and to fully strip lingering epigenetic modulations of the chromatin structure.

The sample was immediately removed from the heat and placed into an ice bath to rapidly cool the sample, preventing unwanted denaturing and to enact the opposite spectrum of the CSP/HSP support which provides critical biological machinery that will rapidly produce temporary lipid rafts and loose membranes around fully primed DNA. At this point the required factors, machinery and chromatin state are fully prepped and the BBNP process gathers elements from the surrounding environment to generate vehicles for preservation and further expansion. The sample is left in the bath for about 3 minutes, long enough to be slightly cold, then brought to room temperature.

The samples were transferred to a growth vessel prepared with a “thin coat” Matrigel. Retinoic acid was added to each separate well/plate. The media used in the protocol with the cells was added to the plate, and supplemented with an additional 2 ml of prepared DMEM media per 1 ml of sample. The cells were then incubated at 37° C. and, 95% humidity, 5% CO₂.

The media was replaced 24 hours later with “core media” which does not contain retinoic acid, but does contain ascorbic acid. Media was changed daily for 72 hours, at which time the media was removed, the cells washed with warm PBS, trypsinized and spun down in centrifuge for 5 mins at 1200 rpm. Gentle titration was used to separate cells and transfer to mouse embryonic fibroblast (MEF) feeders.

At 24 hours, “blastocyst like” cells were gently removed under a hood with microscope assistance and using the smallest micropipette tip possible. The cell and drop of media were transferred to a clean plate. A small drop of trypsin was dropped onto this cell for ˜3 mins to disrupt the outer cells, followed by adding a couple of drops of fresh media to neutralize. The cells were transferred to core media and transferred to incubator. Colonies formed in 3-5 days. Alternatively, the blastocyst like cell may be harvested in the same manner and using a very fine needle, break open the cell, plate and add core media then place in the incubator. Colonies will form within 3-5 days.

Using the methods described above, pluripotent cells and embryos were created from the following species: Human, Mouse, Rat, Chinese Hamster, Pig and Dog. Cell sources included Fibroblasts, Adult and fetal cells, Mesenchymal and hematopoietic cells, Dermal, Immortalized fibroblasts, Adipose tissue, Frozen cell lines, Urine, Cancerous tumor cells, including brain tumor cells.

### Materials and Methods

Core Media. DMEM (Dulbecco's Modified Eagle Medium) high glucose media, supplemented with 1000 μg/ml LIF, 100 μg/ml L-ascorbic acid, 15% Fetal Bovine Serum (FBS), a base medium composed L-glutamine 2 mM, non-essential amino acids 1×, 2-mercaptoethanol lx, Millipore, and penicillin-streptomycin 1×. Special antibiotic treatment−2 ml/500 ml.

Retinoic Acid and L-Ascorbic acid. L-Ascorbic solution was prepared by adding 25 μg/ml DMEM in 15 ml tube. Retinoic Acid solution was prepared by dissolving in DMSO for a concentration of 1 μg/ml for a 15 ml tube.

There are numerous variables dealing with the type of cell being used, density, cell size, etc. It is advantageous to spread two to three samples/wells with a range of the additive chemicals. While almost all the wells will produce colonies, the precise addition is typically hard to define without controlled conditions. Thus, it is likely to see better performance on one end of the scale.

Additionally the protocol may vary with the inclusion of EGF. While the main protocol produces primordial cunctipotent cells, ES cells, pre-embryonic structures and embryoid bodies, the number of cells in each group may vary, and it is therefore advantageous to run all aliquots with gradients of chemical additives.

ATP solution is prepared by measuring 73.4 mg and dissolving in 30 ml PBS in 50 ml tube—then take 150 μl and adding to 15 ml tube with DMEM.

EGF solution is prepared by adding 500 to 15 ml tube with DMEM.

Insulin solution is prepared by adding 0.5 ml to 50 ml tube with DMEM.

### Example 4

Supplementation of embryo culture media with growth factors at: 10 ng/ml EGF, 10 ng/ml IGF-I. These are added to the protocol media on the first day of plating and depending upon the development status, may be discontinued after 48 hours or left for another 24 hours.

Other Growth factor concentrations were 5 ng/ml VEGF, 4 ng/ml Activin A, 10 ng/ml BMP4.

50 embryos were separated out of main culture at 18-24 hours and cultured in 500 ml of media containing 10 ng/ml EGF, 10 ng/ml IGF-1. A second smaller batch of 10 embryos were cultured in 50 ml drops with supplementation.

A second set of the above were prepared with the same media and supplemented with the addition of LIF—this slows the development a little bit but prevents outgrowths.

Cultures may also be grown with only the addition of EGF. It is observed that separating cells into 96 well plates and other tubes with single or few cells results in reduced number of embryos/cells impaired blastocyst formation.

Co-culture of our protocol ES cells with newly forming morula. Use knitting needle to make indentions into petri dishes, cover with KSOM and oil, insert embryo and 5-8 ES cells, incubate for 1-3 hours or until ES cells have attached and then move them all back to “group” environment for blastocyst development.

Similar to blastocyst injection of ES cells to form chimeric mice, the free blastomeres produced during the protocol may produce a co-culture environment for some of the embryos. The disparity between thin TE and normal is likely caused by the competition of overcrowding and failure to initiate Hippo genes and YAP to localize CDx2 to the TE layer.

Early mammalian embryos are highly adaptable during the first three rounds of cleavage and can withstand changes such as the removal, addition, and rearrangement of blastomeres. In the mouse, cells become fully committed to either the TE or ICM lineage during the 32-cell stage, at around E3.5

### Example 5

Passage of cells was performed prior to the growth medium becoming acidic and before the cells reach confluence. For passage, all media was aspirated from the culture vessel and dishes were rinsed with PBS. Room temperature trypsin/Accutase/collagenase was added sufficient to cover the cells. Incubation at room temperature (20° C.) occurred until cells lifted off from the plate and pipetting performed for cell suspension preparation. Cells harvested into 15 ml tubes containing DM EM-15% FBS and centrifuged to pellet the cell suspension (1600 rpm for approximately 6 minutes). Supernatant aspirated and the pellet should be re-suspended in appropriate media, depending upon protocol. Cells should be pipetted until cell pellet disrupted to a single cell suspension or gentle vortexing for embryos before transfer onto a new tissue culture dish.

Following the methods of Example 3, blastocysts were generated. The blastocysts were collected and washed in M2 medium, and transferred onto the prepared MEF feeder layer, Geltrex coated plate or uncoated plate and culture at 37° C. within a 5% CO₂ incubator.

Isolation and dissociation of ICM outgrowth. After 4 to 5 days, gently circle the ICM outgrowths with a finely drawn glass needle, removing the ICM from the surrounding trophoblast cells. Take a sterile non-coated Petri dish and add several small drops (300) of DPBS and Accutase. Transfer the ICM outgrowths to the drops of DPBS, and then repeat this procedure in the Accutase drops and incubate at 37° C. for 15-20 min. Pipette gently, transfer the ICM outgrowths to drops of cell medium and pipet outgrowths into small cell clumps of 5-10 cells. Passage to new plates with desired basement and add media that is supplemented with 10 μM Y-27632. (24 hours then remove and replace media without Y-27632) Change media every other day. For generation of 3 germ layer colonies, the entire outgrowth is lifted with Accutase, spun down and disassociated, then re-plated.

Newly formed ES colonies were collected and dissociated into individual cells using the method above and can be further passaged or spun down and frozen.

### Example 6: Differentiation of Epinul Cells into Neurons

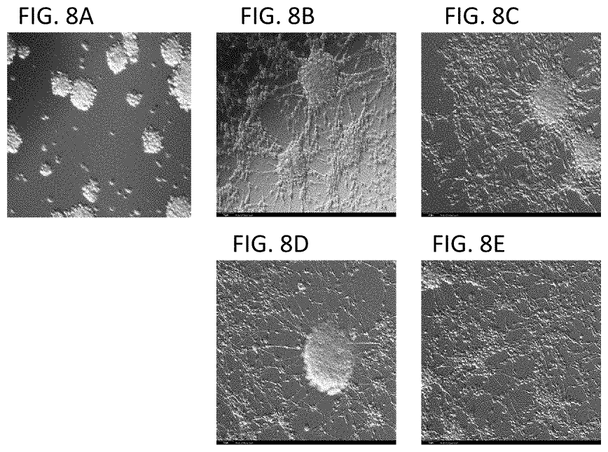

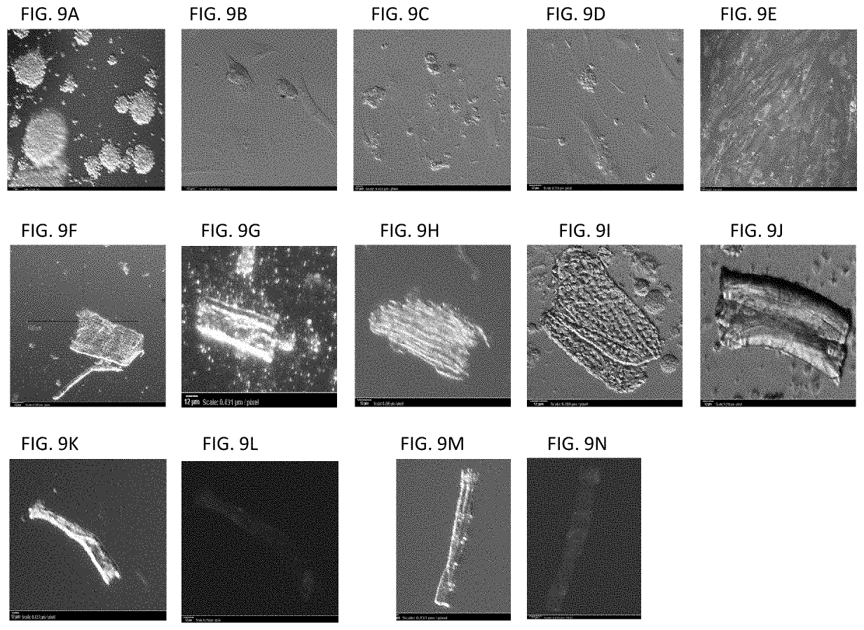

To date the protocol has been employed on human, mouse, rat, porcine and hamster cells. Cells range from foreskin fibroblasts, epithelial, glioblastoma, neuroblastoma, adipose cells, pancreatic cells and ovarian cells. Detailed studies have been done on cells with abnormal karyotypes as well as cancerous cells. The prevailing product from these lines produce normal, healthy pre-embryonic totipotent cells. As the cancerous cells used in these studies appear to be normal and function as a typical cell (growth factor differentiation into new neurons) after protocol and allowing the cells to differentiate through growth factors added to media, the neurons produced remained in normal morphological state, produced new dendrites and interconnected with neighboring cells (FIG. 8). Upon further splitting and passaging, new cell colonies continued to maintain a normal growth pattern. No signs of mutagenesis, tumor formations or clumping could be seen. Well-defined networks were observable.

### Example 7—A Detailed Example of the Janus Protocol

### Materials and Methods

### Post Protocol Growth Media

Media was made with the following volumes:
40% RPMI (Roswell Park Memorial Institute-1640) (200 mL)
40% Dulbecco's modified Eagle's medium (DMEM) with high glucose, pyruvate and L-Glutamine (Gibco, UK) (200 mL)
20% fetal bovine serum (FBS; Gibco, UK) (100 mL)
Add 5 mL β-mercaptoethanol (Sigma, USA) to 0.1 mM
Add 5 mL non-essential amino acids (Sigma, USA) to 0.1 mM
Add 5 mL 100× Penicillin-Streptomycin (10,000 units penicillin and 10 mg streptomycin/mL, sterile-filtered, BioReagent, suitable for cell culture; Sigma-Aldrich)
Add leukemia inhibitory factor (LIF; Sigma, USA) to 1000 U/mL
Add L-Ascorbic Acid to 50 ng/mL
Add CHIR99021 (GSK-3 inhibitor) to 3 μM,
Add Y-27632 (TGF-β RI Kinase Inhibitor II) to 500 nM,
Add PD0325901 (MEK inhibitor) to 1 μM
For human cells, add basic FGF (fibroblast growth factor) to 5 ng/mL
To total volume 500+ mL

Standard Media
450 mL DMEM supplemented with
50 mL fetal bovine serum (to 10%),
Add 5 mL β-mercaptoethanol (Sigma, USA) to 0.1 mM
Add 5 mL non-essential amino acids (Sigma, USA) to 0.1 mM
Add 5 mL 100× Penicillin-Streptomycin (10,000 units penicillin and 10 mg streptomycin/mL, sterile-filtered, BioReagent, suitable for cell culture; Sigma-Aldrich)
Add 5 mL L-glutamine (GlutaMax, ThermoFisher; 200 mM in 0.85% NaCI)
Filter sterilize and store at 4° C. for up to 3-4 weeks.

Stock Solutions for Protocol Step 6:

| | | | Amt. | |
|---|---|---|---|---|
| | Stock | | added | |
| Ingredient | Concentration | Diluent | step 6 | |
| ATP (0.5 mM in PBS; phosphate buffered saline), | 0.5 mM | PBS | 50 | μL/mL |
| EGF (1 μL/mL in PBS), | | | 1 | μL/mL |
| I-ascorbic acid (0.25 μg/mL in PBS), | | | | |
| retinoic acid (0.1 ng/mL DMSO), | | | | |
| 100 × ITS (from Gibco) = insulin | 100x | EBSS | 8 | μL/mL |
| (1 mg/mL), transferrin (0.55 mg/mL) | | | | |
| selenium sodium selenite (0.00067 mg/mL) | | | | |
| (ITS) (3 μL/mL; dilute from 100x soln | | | | |
| l mg/mL prepared in EBSS (Earl's | | | | |
| Balanced Salt Solution) | | | | |
| Proteinase K (0.5 μL/mL) | | | 0.5 | μL/mL |
| NAD (5 μg/mL) | | | 5 | μg/mL |
| CHIR99021 (4, | | | | |
| Y-27632 (25 mM in DMSO), | | | | |
| LIF, | | | | |
| Fetal Bovine Serum (10 μL/mL) | | | 10 | μL/mL |

Cell engineering protocol. Use: somatic cells grown in standard tissue culture media and conditions in T75 tissue culture container to ˜8 million cells. Human fibroblasts are exemplary for this protocol.

Exemplary Steps of an Embodiment of the Janus Protocol:
1. Detach cells with 0.25% trypsin (T75 flask 5 mL trypsin) for ˜5 minutes at room temperature (RT).
2. Transfer to 15 mL conical tube and add 10 mL DMEM to inactivate trypsin digestion.
3. Spin cells down in refrigerated centrifuge at 2000 rpm for 5 mins to pellet.
4. Resuspend cells in 8 mL fresh 4° C. basal DMEM—(cold DMEM retrieved from a 4° refrigerator and kept on ice).
5. Aliquot cells (1 mL in 15 mL tubes×8) (˜1 million cells per 15 mL tube—T75 should give around 8).
6. Add each prepared solution in “Stock solutions for protocol step 6” table to a fresh 15 mL canonical tube with 2 mL (Cold, 4° C.) basal DMEM at specified concentrations in table above appropriate for a total volume of 3 mL (after cell addition in step 7).
7. Transfer suspended cells in 1 mL DMEM into the 15 mL tubes containing the 2 mL DMEM with additives from step 6.
8. Sonicate each 3 mL cell sample using a Qsonica Q700 sonicator with an amplitude set to 37, frequency set between 16 kHz and 20 kHz, power set to 45 W (producing 1,350 j of energy). Probe temperature is set to 65° C. for 17 seconds. Sonication is performed in a series of 3 steps involving sonication and cavitation via insertion of the ⅛″ microprobe into the solution, going from the top, nearly touching the base and returning up and out of the solution over a total time of 5 seconds in three 5 second pulses from 1-5, 7-11, 1217 seconds (2 one second interrupts).
9. During cavitation, observe the solution to see that microbubbles are being generated. The media should become translucent with a noticeable decrease in viscosity. The microbubbles in the solution should dissipate in ˜30 seconds to 1 minute after sonication. A slight media color change should remain.
10. With a strong electronic pipette aide and using a 5 mL pipette, manual cavitation of aliquots is accomplished by flushing samples in and out of the pipette tip rapidly. To accomplish the proper cavitation, shear force, and pressure differentials, tilt the conical tube 45 degrees and insert the pipette tip into the mixture at the opposite angle so the lip on one side of the tip rests against the bottom, leaving a small vertical gap on the rest of the tip—close to but without touching the bottom. Initially pull up forcefully all but the last bit of liquid in the tube—leave enough to cover the tip. It is ok for some air bubbles to get into the pipette. Forcefully expel the solution back into the angle tube (this should produce a vibration that can be felt in the tube you are holding, and the liquid should impact the bottom, swirling around the tube. Repeat this 3 times, then lift the pipette (keeping both tube and pipette angle the same)˜one inch upwards in the solution. Rapidly pull into the pipette one half to two thirds of the liquid and immediately expel back into the remaining liquid. This should be done rapidly, utilizing the full strength of the electronic pipette both drawing and expelling rapidly. If done correctly, by the third or fourth draw, you should begin to see small bubbles and microbubbles forming in the solution. Continue to perform this procedure rapidly until the liquid being drawn into the pipette and the liquid being expelled in the tube becomes cloudy; ˜6-8 draw/expel cycles.
11. Cap the tube and allow it to rest in the hood for ˜1-2 minutes at room temperature.
12. Place conical tube directly into 65° C. water bath, submerging the liquid portion of tube below the water level for 1 minute. Lift the tube and lightly swirl the liquid and place the tube back in the water bath for an additional 15 seconds.
13. Remove tube and place into ice/water bath immediately for 3 minutes with liquid portion below the surface of the ice/water.
14. Remove the samples, lightly swirl the tube and place in rack, sanitize the vials with ethanol and place in the hood.
15. Transfer samples to growth plate(s) at 1 mL per well on a six well plate.
16. Add 1 mL post protocol DMEM media to each 1 mL sample in wells.
17. Incubate at 37° C., 5% CO₂.
18. Depending upon desired cell output, incubation, media changes and additives differ from this point.
19. From this initial period, 24 hours incubation period will result in Cunctipotent cells, early zygote/oocyte and PGC cells, with full expression profiles.
20. Direct derivation and differentiation can be done here, and directed differentiation into cell lineages away from cunctipotent towards functional germ line progenitors can be immediately performed at step 17 (example: addition of Activan A, BMP4.@24 hours, spin down and resuspend in culture media that does not contain the potency factors CHIR, LIF, Y2.) Recipes and protocols for germ line specific derivation are listed on next page.

For assessment of implantation in vitro and further embryonic differentiation, blastocysts were cultured for 8 days. The cell proliferation of outgrowth blastocysts was analyzed by Giemsa staining.

Morphological development was evaluated by light microscopy. Protein expression profiles of single blastocysts were evaluated using antibody stains.

Successful implantation depends on the ability of the embryo to degrade the basement membrane of the uterine epithelium and to invade the uterine stroma. Trophoblast invasion is facilitated by degradation of the extracellular matrix of the endometrium/decidua by various proteinases, among them, the matrix metalloproteinases (MMPs). Successful implantation and trophoblast invasion are closely linked to the expression of MMPs, which are able to degrade basement membranes.

Staining the well after removing the hatched blastocysts allowed visualization of the invasion of cells into the prepared Matrigel plates showed significant invasion and attachment.

Multiple processes control cell lineage specification in the blastocyst to produce the trophoblast, epiblast, and primitive endoderm. These processes include: gene expression, cell signaling, cell-cell contact and positional relationships, and epigenetics. Our embryos undergo full developmental processes and test for nuclear and surface markers associated with the various developmental stages identically to IVF embryos. In other words, fully competent embryos/clone.

Nearly all oocytes expressed pluripotency-related markers, such as stage-specific embryonic antigen-4 (SSEA-4), homeobox gene transcription factor (NANOG), OCT4, and tyrosine kinase receptor for stem cell factor (SCF) (C-KIT)

Once the ICM has been established within the blastocyst, this cell mass prepares for further specification into the epiblast and primitive endoderm. Segregation of blastomeres into the trophoblast and inner cell mass are regulated by the homeodomain protein, Cdx2. These genomic alterations allow for the progressive specification of both epiblast and primitive endoderm lineages at the end of the blastocyst phase of development preceding gastrulation.

### Reagent/Media

Addition of gp130 to culture media improved blastocyst formation.

Growth factors such as human chorionic gonadotropin (hCG) and insulin-like growth factor (IGF)
allow the blastocyst to further invade the endometrium
forskolin and Epidermal Growth Factor (EGF)

### Whole Hatched Blastocyst Derived Fully Pluripotent Embryonic Stem Cells

### Isolated Trophoblast Stem Cells Culture Conditions

### Embryo Development

Follicle-stimulating hormone (FSH), thyroid-stimulating hormone (TSH) and human chorionic gonadotropin (hCG).

Nuff cells (Human foreskin fibroblast/MTI Globalstem) generated embryos via Janus protocol were plated at 24 hours post protocol at (1×10⁴ cells/well) were cultured in 50/50 DMEM/F12 medium containing 10% Fetal Calf Serum, with addition of 1× GlutaMax. Cell culture was maintained in a humidified atmosphere containing 5% CO2 at 37° C. After 24 hours of culture to facilitate cell attachment, the medium was removed, and replaced, harvesting cells with 0.5 ml trypsin to detach, at 80% confluency day three.

The cells were cultured with various concentrations of: a) Forskolin 1-100 μM, b) Epidermal Growth Factor (EGF), 0.8-80 ng/ml (Sigma).

Supplementation of embryo culture media with Growth factors at:
10 ng/mL EGF
10 ng/mL IGF-I
(These are added to the protocol media on the first day of plating and depending upon the development status, may be discontinued after 48 hours or left for another 24 hours.)
Other Growth factor concentrations:
5 ng/mL VEGF
4 ng/mL Activin A
10 ng/mL BMP4

Trophoblast Stem Cells Culture Conditions

FGF2, activin A, XAV939, and Y27632 are sufficient for derivation of TS cells from blastocysts. Undifferentiated TS cell state can be stably maintained in chemically defined culture conditions. Cells expressed TS cell marker genes: Eomes, Elf5, Cdx2, Klf5, Cdh1, Esrrb, Sox2, and Tcfap2c

Differentiated into all trophoblast subtypes (trophoblast giant cells, spongiotrophoblast, and labyrinthine trophoblast) in vitro.

Formation of Embryoid Bodies
1. Trypsinize and dissociate ES cell colonies into single cells.
2. Collect by centrifugation and resuspend the cells at a density of 5×10⁵ cells/ml in Standard medium supplemented with 10 μM Y-27632.
3. Transfer 2 mL cell suspension into a 35 mm dish coated with 1% agar (see Note 5).
4. Culture overnight at incubator, carefully collect cell aggregates and transfer them into a new 35 mm dish coated agar. Add 2 mL MEF medium without Y-27632 for further culture.
5. After 3-5 days, many cystic embryoid bodies appear.

Those skilled in the art can readily recognize that numerous variations and substitutions may be made in the disclosure, its use and its configuration to achieve substantially the same results as achieved by the embodiments described herein.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation and in light of the disclosure, many equivalents to the specific embodiments described herein. It is, therefore, to be understood that the foregoing embodiments are presented by way of example only and that, within the scope of the appended embodiments and equivalents thereto; embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various disclosed concepts may be embodied as one or more methods, of which an example has been provided. The aSEC performed as part of the method may be ordered in any suitable way. Accordingly, embodiments may be constructed in which aSEC are performed in an order different than illustrated, which may include performing some aSEC simultaneously, even though shown as sequential aSEC in illustrative embodiments.

Each document mentioned herein is explicitly incorporated by reference in its entirety. All definitions, as defined and used herein, should be understood to control over dictionary definitions, definitions in documents incorporated by reference (to the extent they differ or conflict), and/or ordinary meanings of the defined terms.

The use of flows or steps is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedia components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

What is claimed is:

1. A method for generating highly potent stem cells from somatic cells by epigenetic conditioning, the method comprising:
subjecting a population of somatic cells to partial protease digestion;
suspending the somatic cells in a medium comprising one or more optional excipients;
exposing the cell suspension to environmental pressures, wherein the environmental pressures comprise electromagnetic disruption of molecular bonds, mechanical stress, heat shock and cold shock, the mechanical stresses being sufficient to subject the cells suspension to an environment characterized by sheer forces, velocity and pressure differentials sufficient to erase epigenetic programming; and
transferring the cells to a growth medium to generate highly potent stem cells comprising totipotent cells.

2. The method of claim 1, wherein the electromagnetic disruption and mechanical stress comprise administration of ultrasonic wave energy sufficient to generate microbubbles in the cell medium subjecting the cells to sheer forces, pressure changes, heat shock, and electromagnetic disruption of molecular bonds.

3. The method of claim 1, wherein the somatic cells are mammalian cells.

4. The method of claim 3, wherein the somatic cells are human cells.

5. The method of claim 1, wherein the Epinul cells are totipotent.

6. The method of claim 1, wherein the Epinul cells are cunctipotent.

7. The method of claim 1, wherein the environmental pressures comprise one or more of ultrasonic, electromagnetic, and thermodynamic disruption, cavitation, pipetting, heat shock and cold shock.

8. The method of claim 1, wherein a series of ultrasonic and pipetting steps are followed by a heat shock step, and a cold shock step.

9. The method of claim 1, wherein the step of applying environmental pressure is performed in medium comprising ATP.

10. The method of claim 1, wherein the Epinul cells are expanded in high glucose culture medium comprising an effective dose of one or more of LIF, retinoic acid, L-ascorbate and EGF.

11. The method of claim 1, wherein the cells that have been exposed to one or more environmental pressures are further treated with proteinase K before the transferring step.

12. A substantially homogenous population of Epinul cells produced by the method of claim 1.

13. A method of generating a differentiated cell population, the method comprising contacting a population of cells according to claim 10 with environmental factors sufficient to induce differentiation.

14. A method for treating or preventing a disease or disorder in a subject in need thereof, comprising generating Epinul cells by the method according to claim 1, wherein the cells are obtained from a subject in need of treatment;
expanding the Epinul cells; and
administering said expanded Epinul cells to the subject in need thereof, wherein said Epinul cells generate differentiated cells that assemble into one or more new tissues or organs following the administration, thereby treating or preventing a disease or disorder in a subject in need thereof.

15. A method for repairing and/or regenerating one or more damaged tissues or organs in a subject in need thereof comprising generating Epinul cells by the method according to claim 1, wherein the cells are obtained from a subject in need of treatment;
expanding the Epinul cells; and
administering said expanded Epinul cells to the subject in need thereof, wherein said Epinul cells generate differentiated cells that assemble into one or more new tissues or organs following the administration, thereby repairing and/or regenerating the one or more damaged tissues or organs.

16. A system for preparing Epinul cells using the method of claim 1.

17. The system of claim 16, wherein the system is automatable and software configurable.

18. The system of claim 17, wherein the system comprises a controlled environment.

19. The system of claim 17, wherein the system comprises chambers.

20. The system of claim 17, wherein the system comprises means to deliver and control environmental pressures.

21. The system of claim 17, wherein the system comprises means to grow and maintain cells.
